SEQUENCE LISTING (35 sequences)

SEQ ID NO: 1 - protein, 915 aa, Pyrococcus horikoshii
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPWAGGGNAGPAFEVLYRGLEVATLVFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSNVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYESHGLTPEIVKEIAEKEGVKVNIPDNFYSMVAKEAERTKEEKGEELVDFELLKDLPDTRRLYYEDPFMKEFDAKVLRVIKDWVILDATAFYPEGGGQPYDTGVLIVNGREVKVTNVQKVGKVIIHKVEDPGAFKEGMIVHGKIDWKRRIQHMRHHTGTHVLMGALVRVLGRHVWQAGSQLTTDWARLDISHYKRISEEELKEIEMLANRIVMEDRKVTWEWLPRTTAEQKYGFRLYQGGVVPGREIRVVKIEDWDVQACGGTHLPSTGLVGPIKILRTERIQDGVERIIFACGEAAIREWQKERDLLKKASNVLRVPPEKLPETAERFFNEWKEARKEVDKLKKELARLLVYELESKMQKIGSIEFIGEVVEGSMEDLRELVEKLKKPKRVVVLISRDGYFAVSVGSEVGVEANELAKKITLIAGGGGGGRRDIAQGKVKDISKAKDVIESIKSMFS

SEQ ID NO: 2 - DNA, 78 bp, Pyrococcus horikoshii
  source 1..78: mol_type = other DNA
gggccggtagctcagcctggtatgagcgccgcccttgcaaggcggaggccccgggttcaaatcccggccggtccacca

SEQ ID NO: 3 - DNA, 78 bp, synthetic construct
  misc_feature 1..78: note = polypeptide
  source 1..78: mol_type = other DNA
gggccggtagctcagcctggtatgagcgccgccctctaaaggcggaggccccgggttcaaatcccggccggtccacca

SEQ ID NO: 4 - DNA, 78 bp, synthetic construct
  misc_feature 1..78: note = polypeptide
  source 1..78: mol_type = other DNA
ggaccggtagctcagcctggtatgagcgccgccctctaaaggcggaggccccgggttcaaatcccggccggtccacca

SEQ ID NO: 5 - protein, 149 aa, synthetic construct
  REGION 1..149: note = polypeptide
  SITE 3: note = MISC_FEATURE - Aib
MSXVPIQKVQDDTKTLIKTIVTRINDISHTQSVSSKQKVTGLDFIPGLHPILTLSKMDQTLAVYQQILTSMPSRNVIQISNDLENLRDLLHVLAFSKSCHLPWASGLETLDSLGGVLEASGYSTEVVALSRLQGSLQDMLWQLDLSPGC

SEQ ID NO: 6 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPWAGGGNAGPAFEVLYRGLEVATLVFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSNVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYESHGLTPEIVKEIAEKEGVKVNIPDNFYSMVAKEAER

SEQ ID NO: 7 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPWAGGGNAGPAFEVLYRGLEVATLGFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSNVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYESHGLTPEIVKEIAEKEGVKVNIPDNFYSMVAKEAER

SEQ ID NO: 8 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPFAGGGNAGPAFEVLYRGLEVATLVFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSNVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYESHGLTPEIVKEIAEKEGVKVNIPDNFYSMVAKEAER

SEQ ID NO: 9 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVATLVFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSNVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYESHGLTPEIVKEIAEKEGVKVNIPDNFYSMVAKEAER

SEQ ID NO: 10 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVATLGFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSNVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYESHGLTPEIVKEIAEKEGVKVNIPDNFYSMVAKEAER

SEQ ID NO: 11 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVATLGFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 12 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVATLGFIQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 13 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVATLGFLQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 14 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHLGGGNAGPAFEVLYRGLEVATLGFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 15 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVATLGWMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 16 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHLGGGNAGPAFEVLYRGLEVATLGWMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 17 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHIGGGNAGPAFEVLYRGLEVATLGFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 18 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHIGGGNAGPAFEVLYRGLEVATLGFIQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 19 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHLGGGNAGPAFEVLYRGLEVATLGFIQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 20 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHIGGGNAGPAFEVLYRGLEVATLGFLQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 21 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHLGGGNAGPAFEVLYRGLEVATLGFLQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 22 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPVAGGGNAGPAFEVLYRGLEVATLGFIQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 23 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPIAGGGNAGPAFEVLYRGLEVATLGFIQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 24 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPLAGGGNAGPAFEVLYRGLEVATLGFIQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 25 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHGGGGNAGPAFEVLYRGLEVATLGFIQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 26 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVASLGFMQYKKAPENAPQDQVVVIKGEKYIPMETKVVDTGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 27 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVATLGFMQYKKAPENAPQDQVVVIKGEKYIPMETKVVDSGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 28 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVATLGFMQYKKAPENAPQDQVVVIKGEKYIPMETKVVDVGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 29 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVACLGFMQYKKAPENAPQDQVVVIKGEKYIPMETKVVDVGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 30 - protein, 495 aa, synthetic construct
  REGION 1..495: note = polypeptide
MEFIMKTRMFEEEGWIRKKCKVCGKPFWTLDPDRETCGDPPCDEYQFIGKPGIPRKYTLDEMREKFLRFFEKHEIYPHGRVKRYPVLPRWRDDVLLVGASIMDFQPWVISGEADPPANPLVISQPSIRFTDIDNVGITGRHFTIFEMMAHHAFNYPGKPIYWMDETVELAFEFFTKELKMKPEDITFKENPHAGGGNAGPAFEVLYRGLEVAALGFMQYKKAPENAPQDQVVVIKGEKYIPMETKVVDFGYGLERLVWMSQGTPTAYDAVLGYVVEPLKKMAGIEKIDEKILMENSRLAGMFDIEDLGDLRYLREQVAKRVGITVEELEKAIRPYELIYAIADHTKALTFMLADGVVPSAVKAGYLARLLIRKSIRHLRELGLEVPLSEIVALHIKELHKTFPEFKEMEDIILEMIELEEKKYAETLRRGSDLVRREIAKLKKKGIKEIPVEKLVTFYASHGLTPEIVKEIAEKEGVKVNIPDNFYSMVAKEAER

SEQ ID NO: 31 - protein, 75 aa, synthetic construct
  REGION 1..75: note = polypeptide
  SITE 46: note = MISC_FEATURE - Aib
MHKSSPQGPDRLLIRLRHLIDIVESKSRSKSRASGSDVKDDGDRHXEGTFTSDVSSYLEGQAAKEFIAWLVRGRG

SEQ ID NO: 32 - protein, 31 aa, synthetic construct
  REGION 1..31: note = polypeptide
  SITE 2: note = MISC_FEATURE - Aib
  SITE 20: note = 18-[[(1S)-4-[2-[2-[2-[2-[2-[2-[[(5S)-5-amino-5-carb oxy-pentyl]amino]-2-oxo-ethoxy]ethoxy]ethylamino]-2-oxo-et hoxy]ethoxy]ethylamino]-1-carboxy-4-oxo-butyl]amino]-18-ox o-octadecanoic acid modified Lysine
HXEGTFTSDVSSYLEGQAAKEFIAWLVRGRG

SEQ ID NO: 33 - protein, 494 aa, synthetic construct
  REGION 1..494: note = Formula I
  VARIANT 1: note = Any amino acid or absent
  VARIANT 2: note = Any amino acid or absent
  VARIANT 3: note = Any amino acid or absent
  VARIANT 4: note = Any amino acid or absent
  VARIANT 5: note = Any amino acid or absent
  VARIANT 6: note = Any amino acid or absent
  VARIANT 7: note = Any amino acid or absent
  VARIANT 8: note = Any amino acid or absent
  VARIANT 9: note = Any amino acid or absent
  VARIANT 11: note = Any amino acid or absent
  VARIANT 13: note = Any amino acid or absent
  VARIANT 15: note = Any amino acid or absent
  VARIANT 16: note = Any amino acid or absent
  VARIANT 17: note = Any amino acid or absent
  VARIANT 19: note = Any amino acid or absent
  VARIANT 21: note = Any amino acid or absent
  VARIANT 22: note = Any amino acid or absent
  VARIANT 25: note = Any amino acid or absent
  VARIANT 26: note = Any amino acid or absent
  VARIANT 30: note = Any amino acid or absent
  VARIANT 31: note = Any amino acid or absent
  VARIANT 32: note = Any amino acid or absent
  VARIANT 33: note = Any amino acid or absent
  VARIANT 35: note = Any amino acid or absent
  VARIANT 36: note = Any amino acid or absent
  VARIANT 40: note = Any amino acid or absent
  VARIANT 43: note = Any amino acid or absent
  VARIANT 44: note = Any amino acid or absent
  VARIANT 46: note = Any amino acid or absent
  VARIANT 50: note = Any amino acid or absent
  VARIANT 52: note = Any amino acid or absent
  VARIANT 53: note = Any amino acid or absent
  VARIANT 54: note = Any amino acid or absent
  VARIANT 55: note = Any amino acid or absent
  VARIANT 56: note = Any amino acid or absent
  VARIANT 57: note = Any amino acid or absent
  VARIANT 58: note = Any amino acid or absent
  VARIANT 60: note = Any amino acid or absent
  VARIANT 62: note = Any amino acid or absent
  VARIANT 64: note = Any amino acid or absent
  VARIANT 65: note = Any amino acid or absent
  VARIANT 67: note = Any amino acid or absent
  VARIANT 68: note = Any amino acid or absent
  VARIANT 72: note = Any amino acid or absent
  VARIANT 73: note = Any amino acid or absent
  VARIANT 74: note = Any amino acid or absent
  VARIANT 76: note = Any amino acid or absent
  VARIANT 77: note = Any amino acid or absent
  VARIANT 78: note = Any amino acid or absent
  VARIANT 79: note = Any amino acid or absent
  VARIANT 84: note = Any amino acid or absent
  VARIANT 85: note = Any amino acid or absent
  VARIANT 92: note = Any amino acid or absent
  VARIANT 99: note = Any amino acid or absent
  VARIANT 106: note = Any amino acid or absent
  VARIANT 107: note = Any amino acid or absent
  VARIANT 109: note = Any amino acid or absent
  VARIANT 110: note = Any amino acid or absent
  VARIANT 111: note = Any amino acid or absent
  VARIANT 118: note = Any amino acid or absent
  VARIANT 123: note = Any amino acid or absent
  VARIANT 126: note = Any amino acid or absent
  VARIANT 127: note = Any amino acid or absent
  VARIANT 129: note = Any amino acid or absent
  VARIANT 131: note = Any amino acid or absent
  VARIANT 134: note = Any amino acid or absent
  VARIANT 135: note = Any amino acid or absent
  VARIANT 139: note = Any amino acid or absent
  VARIANT 141: note = Any amino acid or absent
  VARIANT 152: note = Any amino acid or absent
  VARIANT 153: note = Any amino acid or absent
  VARIANT 154: note = Any amino acid or absent
  VARIANT 155: note = Any amino acid or absent
  VARIANT 156: note = Any amino acid or absent
  VARIANT 157: note = Any amino acid or absent
  VARIANT 160: note = Any amino acid or absent
  VARIANT 161: note = Any amino acid or absent
  VARIANT 165: note = Any amino acid or absent
  VARIANT 166: note = Any amino acid or absent
  VARIANT 167: note = Any amino acid or absent
  VARIANT 168: note = Any amino acid or absent
  VARIANT 169: note = Any amino acid or absent
  VARIANT 171: note = Any amino acid or absent
  VARIANT 172: note = Any amino acid or absent
  VARIANT 173: note = Any amino acid or absent
  VARIANT 174: note = Any amino acid or absent
  VARIANT 175: note = Any amino acid or absent
  VARIANT 176: note = Any amino acid or absent
  VARIANT 177: note = Any amino acid or absent
  VARIANT 178: note = Any amino acid or absent
  VARIANT 179: note = Any amino acid or absent
  VARIANT 180: note = Any amino acid or absent
  VARIANT 181: note = Any amino acid or absent
  VARIANT 185: note = Any amino acid or absent
  VARIANT 187: note = Any amino acid or absent
  VARIANT 188: note = Any amino acid or absent
  VARIANT 190: note = Any amino acid or absent
  VARIANT 197: note = Any amino acid or absent
  VARIANT 198: note = Any amino acid or absent
  VARIANT 199: note = Any amino acid or absent
  VARIANT 202: note = Any amino acid or absent
  VARIANT 203: note = Any amino acid or absent
  VARIANT 204: note = Any amino acid or absent
  VARIANT 206: note = Any amino acid or absent
  VARIANT 208: note = Any amino acid or absent
  VARIANT 215: note = Any amino acid or absent
  VARIANT 217: note = Any amino acid or absent
  VARIANT 218: note = Any amino acid or absent
  VARIANT 219: note = Any amino acid or absent
  VARIANT 220: note = Any amino acid or absent
  VARIANT 221: note = Any amino acid or absent
  VARIANT 222: note = Any amino acid or absent
  VARIANT 223: note = Any amino acid or absent
  VARIANT 224: note = Any amino acid or absent
  VARIANT 225: note = Any amino acid or absent
  VARIANT 226: note = Any amino acid or absent
  VARIANT 227: note = Any amino acid or absent
  VARIANT 228: note = Any amino acid or absent
  VARIANT 229: note = Any amino acid or absent
  VARIANT 230: note = Any amino acid or absent
  VARIANT 231: note = Any amino acid or absent
  VARIANT 232: note = Any amino acid or absent
  VARIANT 233: note = Any amino acid or absent
  VARIANT 234: note = Any amino acid or absent
  VARIANT 235: note = Any amino acid or absent
  VARIANT 236: note = Any amino acid or absent
  VARIANT 237: note = Any amino acid or absent
  VARIANT 239: note = Any amino acid or absent
  VARIANT 240: note = Any amino acid or absent
  VARIANT 241: note = Any amino acid or absent
  VARIANT 242: note = Any amino acid or absent
  VARIANT 243: note = Any amino acid or absent
  VARIANT 253: note = Any amino acid or absent
  VARIANT 254: note = Any amino acid or absent
  VARIANT 256: note = Any amino acid or absent
  VARIANT 258: note = Any amino acid or absent
  VARIANT 260: note = Any amino acid or absent
  VARIANT 263: note = Any amino acid or absent
  VARIANT 267: note = Any amino acid or absent
  VARIANT 268: note = Any amino acid or absent
  VARIANT 269: note = Any amino acid or absent
  VARIANT 270: note = Any amino acid or absent
  VARIANT 271: note = Any amino acid or absent
  VARIANT 272: note = Any amino acid or absent
  VARIANT 273: note = Any amino acid or absent
  VARIANT 274: note = Any amino acid or absent
  VARIANT 276: note = Any amino acid or absent
  VARIANT 277: note = Any amino acid or absent
  VARIANT 278: note = Any amino acid or absent
  VARIANT 281: note = Any amino acid or absent
  VARIANT 282: note = Any amino acid or absent
  VARIANT 283: note = Any amino acid or absent
  VARIANT 284: note = Any amino acid or absent
  VARIANT 285: note = Any amino acid or absent
  VARIANT 286: note = Any amino acid or absent
  VARIANT 287: note = Any amino acid or absent
  VARIANT 288: note = Any amino acid or absent
  VARIANT 291: note = Any amino acid or absent
  VARIANT 292: note = Any amino acid or absent
  VARIANT 293: note = Any amino acid or absent
  VARIANT 294: note = Any amino acid or absent
  VARIANT 295: note = Any amino acid or absent
  VARIANT 296: note = Any amino acid or absent
  VARIANT 299: note = Any amino acid or absent
  VARIANT 300: note = Any amino acid or absent
  VARIANT 302: note = Any amino acid or absent
  VARIANT 303: note = Any amino acid or absent
  VARIANT 304: note = Any amino acid or absent
  VARIANT 305: note = Any amino acid or absent
  VARIANT 306: note = Any amino acid or absent
  VARIANT 307: note = Any amino acid or absent
  VARIANT 309: note = Any amino acid or absent
  VARIANT 310: note = Any amino acid or absent
  VARIANT 313: note = Any amino acid or absent
  VARIANT 314: note = Any amino acid or absent
  VARIANT 317: note = Any amino acid or absent
  VARIANT 318: note = Any amino acid or absent
  VARIANT 319: note = Any amino acid or absent
  VARIANT 320: note = Any amino acid or absent
  VARIANT 321: note = Any amino acid or absent
  VARIANT 322: note = Any amino acid or absent
  VARIANT 323: note = Any amino acid or absent
  VARIANT 324: note = Any amino acid or absent
  VARIANT 327: note = Any amino acid or absent
  VARIANT 328: note = Any amino acid or absent
  VARIANT 329: note = Any amino acid or absent
  VARIANT 330: note = Any amino acid or absent
  VARIANT 331: note = Any amino acid or absent
  VARIANT 333: note = Any amino acid or absent
  VARIANT 335: note = Any amino acid or absent
  VARIANT 336: note = Any amino acid or absent
  VARIANT 344: note = Any amino acid or absent
  VARIANT 345: note = Any amino acid or absent
  VARIANT 347: note = Any amino acid or absent
  VARIANT 351: note = Any amino acid or absent
  VARIANT 355: note = Any amino acid or absent
  VARIANT 359: note = Any amino acid or absent
  VARIANT 360: note = Any amino acid or absent
  VARIANT 368: note = Any amino acid or absent
  VARIANT 371: note = Any amino acid or absent
  VARIANT 372: note = Any amino acid or absent
  VARIANT 373: note = Any amino acid or absent
  VARIANT 375: note = Any amino acid or absent
  VARIANT 376: note = Any amino acid or absent
  VARIANT 377: note = Any amino acid or absent
  VARIANT 378: note = Any amino acid or absent
  VARIANT 381: note = Any amino acid or absent
  VARIANT 382: note = Any amino acid or absent
  VARIANT 383: note = Any amino acid or absent
  VARIANT 386: note = Any amino acid or absent
  VARIANT 387: note = Any amino acid or absent
  VARIANT 389: note = Any amino acid or absent
  VARIANT 390: note = Any amino acid or absent
  VARIANT 391: note = Any amino acid or absent
  VARIANT 392: note = Any amino acid or absent
  VARIANT 393: note = Any amino acid or absent
  VARIANT 394: note = Any amino acid or absent
  VARIANT 395: note = Any amino acid or absent
  VARIANT 396: note = Any amino acid or absent
  VARIANT 397: note = Any amino acid or absent
  VARIANT 398: note = Any amino acid or absent
  VARIANT 399: note = Any amino acid or absent
  VARIANT 400: note = Any amino acid or absent
  VARIANT 403: note = Any amino acid or absent
  VARIANT 404: note = Any amino acid or absent
  VARIANT 406: note = Any amino acid or absent
  VARIANT 407: note = Any amino acid or absent
  VARIANT 408: note = Any amino acid or absent
  VARIANT 409: note = Any amino acid or absent
  VARIANT 410: note = Any amino acid or absent
  VARIANT 411: note = Any amino acid or absent
  VARIANT 412: note = Any amino acid or absent
  VARIANT 413: note = Any amino acid or absent
  VARIANT 414: note = Any amino acid or absent
  VARIANT 415: note = Any amino acid or absent
  VARIANT 416: note = Any amino acid or absent
  VARIANT 419: note = Any amino acid or absent
  VARIANT 420: note = Any amino acid or absent
  VARIANT 421: note = Any amino acid or absent
  VARIANT 422: note = Any amino acid or absent
  VARIANT 423: note = Any amino acid or absent
  VARIANT 425: note = Any amino acid or absent
  VARIANT 426: note = Any amino acid or absent
  VARIANT 429: note = Any amino acid or absent
  VARIANT 430: note = Any amino acid or absent
  VARIANT 431: note = Any amino acid or absent
  VARIANT 432: note = Any amino acid or absent
  VARIANT 433: note = Any amino acid or absent
  VARIANT 434: note = Any amino acid or absent
  VARIANT 435: note = Any amino acid or absent
  VARIANT 436: note = Any amino acid or absent
  VARIANT 437: note = Any amino acid or absent
  VARIANT 438: note = Any amino acid or absent
  VARIANT 439: note = Any amino acid or absent
  VARIANT 441: note = Any amino acid or absent
  VARIANT 442: note = Any amino acid or absent
  VARIANT 444: note = Any amino acid or absent
  VARIANT 445: note = Any amino acid or absent
  VARIANT 446: note = Any amino acid or absent
  VARIANT 447: note = Any amino acid or absent
  VARIANT 449: note = Any amino acid or absent
  VARIANT 450: note = Any amino acid or absent
  VARIANT 451: note = Any amino acid or absent
  VARIANT 453: note = Any amino acid or absent
  VARIANT 454: note = Any amino acid or absent
  VARIANT 455: note = Any amino acid or absent
  VARIANT 457: note = Any amino acid or absent
  VARIANT 461: note = Any amino acid or absent
  VARIANT 462: note = Any amino acid or absent
  VARIANT 465: note = Any amino acid or absent
  VARIANT 466: note = Any amino acid or absent
  VARIANT 467: note = Any amino acid or absent
  VARIANT 469: note = Any amino acid or absent
  VARIANT 471: note = Any amino acid or absent
  VARIANT 472: note = Any amino acid or absent
  VARIANT 473: note = Any amino acid or absent
  VARIANT 474: note = Any amino acid or absent
  VARIANT 475: note = Any amino acid or absent
  VARIANT 476: note = Any amino acid or absent
  VARIANT 478: note = Any amino acid or absent
  VARIANT 479: note = Any amino acid or absent
  VARIANT 485: note = Any amino acid or absent
  VARIANT 486: note = Any amino acid or absent
  VARIANT 489: note = Any amino acid or absent
  VARIANT 490: note = Any amino acid or absent
  VARIANT 491: note = Any amino acid or absent
  VARIANT 492: note = Any amino acid or absent
  VARIANT 493: note = Any amino acid or absent
  VARIANT 494: note = Any amino acid or absent
XXXXXXXXXFXEXGXXXKXCXXCGXXFWTXXXXRXXCGDXPCXXYXFIGXPXXXXXXXLXEXRXXFXXFFEXXXHXXXXRYPVXXRWRDDVXLVGASIXDFQPWVXXGXXXPPANPLXISQPXIRXXDXDXVGXXGRHXTXFEMMAHHAFNXXXXXXYWXXETVXXXXXFXXXXXXXXXXXITFXEXXWXGGGNAGXXXEVXXXGXEXATLVFMXYXXXXXXXXXXXXXXXXXXXXXPXXXXXVDTGYGLERXXWXSXGXPTXYDAXXXXXXXXLXXXAGXXXXXXXXILXXXXXXAGXXDXXXXXXLXXLRXXVAXXXXXXXXELXXXXXPXEXXYAIADHTXXLXFMLXDGVXPSNXXAGYLARLXIRXXXRXXXXLGXXXPLXXIXXXXXXXXXXXXPEXXEXXXXXXXXXXXEEXXXXXTXXRGXXXXXXXXXXXKXXGXXXXPXXXLXXXYXSHGXXPEXXXEXAXXXXXXVXXPDNFYXXVAXXXXXX

SEQ ID NO: 34 - protein, 494 aa, synthetic construct
  REGION 1..494: note = Formula II
  VARIANT 1: note = Any amino acid or absent
  VARIANT 2: note = Any amino acid or absent
  VARIANT 3: note = Any amino acid or absent
  VARIANT 4: note = Any amino acid or absent
  VARIANT 5: note = Any amino acid or absent
  VARIANT 6: note = Any amino acid or absent
  VARIANT 7: note = Any amino acid or absent
  VARIANT 8: note = Any amino acid or absent
  VARIANT 9: note = Any amino acid or absent
  VARIANT 11: note = Any amino acid or absent
  VARIANT 13: note = Any amino acid or absent
  VARIANT 15: note = Any amino acid or absent
  VARIANT 16: note = Any amino acid or absent
  VARIANT 19: note = Any amino acid or absent
  VARIANT 21: note = Any amino acid or absent
  VARIANT 22: note = Any amino acid or absent
  VARIANT 25: note = Any amino acid or absent
  VARIANT 26: note = Any amino acid or absent
  VARIANT 30: note = Any amino acid or absent
  VARIANT 40: note = Any amino acid or absent
  VARIANT 44: note = Any amino acid or absent
  VARIANT 46: note = Any amino acid or absent
  VARIANT 50: note = Any amino acid or absent
  VARIANT 52: note = Any amino acid or absent
  VARIANT 53: note = Any amino acid or absent
  VARIANT 54: note = Any amino acid or absent
  VARIANT 55: note = Any amino acid or absent
  VARIANT 56: note = Any amino acid or absent
  VARIANT 58: note = Any amino acid or absent
  VARIANT 60: note = Any amino acid or absent
  VARIANT 62: note = Any amino acid or absent
  VARIANT 65: note = Any amino acid or absent
  VARIANT 68: note = Any amino acid or absent
  VARIANT 72: note = Any amino acid or absent
  VARIANT 73: note = Any amino acid or absent
  VARIANT 74: note = Any amino acid or absent
  VARIANT 76: note = Any amino acid or absent
  VARIANT 78: note = Any amino acid or absent
  VARIANT 79: note = Any amino acid or absent
  VARIANT 84: note = Any amino acid or absent
  VARIANT 85: note = Any amino acid or absent
  VARIANT 92: note = Any amino acid or absent
  VARIANT 99: note = Any amino acid or absent
  VARIANT 106: note = Any amino acid or absent
  VARIANT 109: note = Any amino acid or absent
  VARIANT 110: note = Any amino acid or absent
  VARIANT 111: note = Any amino acid or absent
  VARIANT 118: note = Any amino acid or absent
  VARIANT 126: note = Any amino acid or absent
  VARIANT 129: note = Any amino acid or absent
  VARIANT 134: note = Any amino acid or absent
  VARIANT 139: note = Any amino acid or absent
  VARIANT 141: note = Any amino acid or absent
  VARIANT 152: note = Any amino acid or absent
  VARIANT 154: note = Any amino acid or absent
  VARIANT 155: note = Any amino acid or absent
  VARIANT 156: note = Any amino acid or absent
  VARIANT 157: note = Any amino acid or absent
  VARIANT 160: note = Any amino acid or absent
  VARIANT 166: note = Any amino acid or absent
  VARIANT 167: note = Any amino acid or absent
  VARIANT 168: note = Any amino acid or absent
  VARIANT 169: note = Any amino acid or absent
  VARIANT 171: note = Any amino acid or absent
  VARIANT 173: note = Any amino acid or absent
  VARIANT 174: note = Any amino acid or absent
  VARIANT 176: note = Any amino acid or absent
  VARIANT 177: note = Any amino acid or absent
  VARIANT 178: note = Any amino acid or absent
  VARIANT 179: note = Any amino acid or absent
  VARIANT 181: note = Any amino acid or absent
  VARIANT 187: note = Any amino acid or absent
  VARIANT 188: note = Any amino acid or absent
  VARIANT 190: note = Any amino acid or absent
  VARIANT 198: note = Any amino acid or absent
  VARIANT 199: note = Any amino acid or absent
  VARIANT 203: note = Any amino acid or absent
  VARIANT 218: note = Any amino acid or absent
  VARIANT 219: note = Any amino acid or absent
  VARIANT 220: note = Any amino acid or absent
  VARIANT 221: note = Any amino acid or absent
  VARIANT 222: note = Any amino acid or absent
  VARIANT 223: note = Any amino acid or absent
  VARIANT 224: note = Any amino acid or absent
  VARIANT 225: note = Any amino acid or absent
  VARIANT 226: note = Any amino acid or absent
  VARIANT 227: note = Any amino acid or absent
  VARIANT 228: note = Any amino acid or absent
  VARIANT 229: note = Any amino acid or absent
  VARIANT 230: note = Any amino acid or absent
  VARIANT 231: note = Any amino acid or absent
  VARIANT 232: note = Any amino acid or absent
  VARIANT 234: note = Any amino acid or absent
  VARIANT 235: note = Any amino acid or absent
  VARIANT 236: note = Any amino acid or absent
  VARIANT 237: note = Any amino acid or absent
  VARIANT 240: note = Any amino acid or absent
  VARIANT 241: note = Any amino acid or absent
  VARIANT 242: note = Any amino acid or absent
  VARIANT 243: note = Any amino acid or absent
  VARIANT 253: note = Any amino acid or absent
  VARIANT 256: note = Any amino acid or absent
  VARIANT 258: note = Any amino acid or absent
  VARIANT 268: note = Any amino acid or absent
  VARIANT 269: note = Any amino acid or absent
  VARIANT 270: note = Any amino acid or absent
  VARIANT 272: note = Any amino acid or absent
  VARIANT 273: note = Any amino acid or absent
  VARIANT 274: note = Any amino acid or absent
  VARIANT 276: note = Any amino acid or absent
  VARIANT 277: note = Any amino acid or absent
  VARIANT 278: note = Any amino acid or absent
  VARIANT 281: note = Any amino acid or absent
  VARIANT 282: note = Any amino acid or absent
  VARIANT 283: note = Any amino acid or absent
  VARIANT 284: note = Any amino acid or absent
  VARIANT 285: note = Any amino acid or absent
  VARIANT 286: note = Any amino acid or absent
  VARIANT 287: note = Any amino acid or absent
  VARIANT 288: note = Any amino acid or absent
  VARIANT 291: note = Any amino acid or absent
  VARIANT 295: note = Any amino acid or absent
  VARIANT 299: note = Any amino acid or absent
  VARIANT 300: note = Any amino acid or absent
  VARIANT 304: note = Any amino acid or absent
  VARIANT 305: note = Any amino acid or absent
  VARIANT 306: note = Any amino acid or absent
  VARIANT 309: note = Any amino acid or absent
  VARIANT 310: note = Any amino acid or absent
  VARIANT 313: note = Any amino acid or absent
  VARIANT 314: note = Any amino acid or absent
  VARIANT 317: note = Any amino acid or absent
  VARIANT 318: note = Any amino acid or absent
  VARIANT 319: note = Any amino acid or absent
  VARIANT 322: note = Any amino acid or absent
  VARIANT 323: note = Any amino acid or absent
  VARIANT 324: note = Any amino acid or absent
  VARIANT 327: note = Any amino acid or absent
  VARIANT 328: note = Any amino acid or absent
  VARIANT 329: note = Any amino acid or absent
  VARIANT 330: note = Any amino acid or absent
  VARIANT 331: note = Any amino acid or absent
  VARIANT 333: note = Any amino acid or absent
  VARIANT 335: note = Any amino acid or absent
  VARIANT 344: note = Any amino acid or absent
  VARIANT 345: note = Any amino acid or absent
  VARIANT 347: note = Any amino acid or absent
  VARIANT 351: note = Any amino acid or absent
  VARIANT 355: note = Any amino acid or absent
  VARIANT 371: note = Any amino acid or absent
  VARIANT 372: note = Any amino acid or absent
  VARIANT 373: note = Any amino acid or absent
  VARIANT 375: note = Any amino acid or absent
  VARIANT 376: note = Any amino acid or absent
  VARIANT 377: note = Any amino acid or absent
  VARIANT 378: note = Any amino acid or absent
  VARIANT 382: note = Any amino acid or absent
  VARIANT 383: note = Any amino acid or absent
  VARIANT 386: note = Any amino acid or absent
  VARIANT 389: note = Any amino acid or absent
  VARIANT 390: note = Any amino acid or absent
  VARIANT 391: note = Any amino acid or absent
  VARIANT 392: note = Any amino acid or absent
  VARIANT 393: note = Any amino acid or absent
  VARIANT 394: note = Any amino acid or absent
  VARIANT 395: note = Any amino acid or absent
  VARIANT 397: note = Any amino acid or absent
  VARIANT 398: note = Any amino acid or absent
  VARIANT 399: note = Any amino acid or absent
  VARIANT 400: note = Any amino acid or absent
  VARIANT 403: note = Any amino acid or absent
  VARIANT 404: note = Any amino acid or absent
  VARIANT 406: note = Any amino acid or absent
  VARIANT 407: note = Any amino acid or absent
  VARIANT 408: note = Any amino acid or absent
  VARIANT 409: note = Any amino acid or absent
  VARIANT 411: note = Any amino acid or absent
  VARIANT 412: note = Any amino acid or absent
  VARIANT 413: note = Any amino acid or absent
  VARIANT 414: note = Any amino acid or absent
  VARIANT 415: note = Any amino acid or absent
  VARIANT 416: note = Any amino acid or absent
  VARIANT 419: note = Any amino acid or absent
  VARIANT 420: note = Any amino acid or absent
  VARIANT 422: note = Any amino acid or absent
  VARIANT 423: note = Any amino acid or absent
  VARIANT 426: note = Any amino acid or absent
  VARIANT 429: note = Any amino acid or absent
  VARIANT 430: note = Any amino acid or absent
  VARIANT 433: note = Any amino acid or absent
  VARIANT 434: note = Any amino acid or absent
  VARIANT 435: note = Any amino acid or absent
  VARIANT 436: note = Any amino acid or absent
  VARIANT 437: note = Any amino acid or absent
  VARIANT 438: note = Any amino acid or absent
  VARIANT 442: note = Any amino acid or absent
  VARIANT 444: note = Any amino acid or absent
  VARIANT 445: note = Any amino acid or absent
  VARIANT 447: note = Any amino acid or absent
  VARIANT 451: note = Any amino acid or absent
  VARIANT 453: note = Any amino acid or absent
  VARIANT 454: note = Any amino acid or absent
  VARIANT 455: note = Any amino acid or absent
  VARIANT 457: note = Any amino acid or absent
  VARIANT 461: note = Any amino acid or absent
  VARIANT 462: note = Any amino acid or absent
  VARIANT 465: note = Any amino acid or absent
  VARIANT 467: note = Any amino acid or absent
  VARIANT 469: note = Any amino acid or absent
  VARIANT 471: note = Any amino acid or absent
  VARIANT 472: note = Any amino acid or absent
  VARIANT 473: note = Any amino acid or absent
  VARIANT 475: note = Any amino acid or absent
  VARIANT 476: note = Any amino acid or absent
  VARIANT 478: note = Any amino acid or absent
  VARIANT 479: note = Any amino acid or absent
  VARIANT 485: note = Any amino acid or absent
  VARIANT 489: note = Any amino acid or absent
  VARIANT 490: note = Any amino acid or absent
  VARIANT 491: note = Any amino acid or absent
  VARIANT 493: note = Any amino acid or absent
  VARIANT 494: note = Any amino acid or absent
XXXXXXXXXFXEXGXXRKXCXXCGXXFWTXDPDRETCGDXPCDXYXFIGXPXXXXXYXLXEXREXFLXFFEXXXHXRXXRYPVXXRWRDDVXLVGASIXDFQPWVXSGXXXPPANPLXISQPSIRXTDXDNVGXTGRHXTXFEMMAHHAFNXPXXXXYWXDETVEXXXXFXTXXLXXXXEXITFKEXXWXGGGNAGPXXEVLXRGLEVATLVFMQYKXXXXXXXXXXXXXXXGXXXXPMXXXXVDTGYGLERXVWXSXGTPTAYDAVXXXVXXXLXXXAGXXXXXXXXILXENSXLAGXXDIEXXXDLXXLRXXVAXXXGIXXXELXXXXXPXEXIYAIADHTXXLXFMLXDGVXPSNVKAGYLARLLIRXXXRXXXXLGLXXPLXEIXXXXXXXLXXXXPEXXEXXXXIXXXXXXEEXXYXXTLXRGXXLVXXXXXXLKKXGXXEXPLEXLXXXYXSHGXXPEXVXEXAXXXGXXVXXPDNFYXLVAXXXEXX

SEQ ID NO: 35 - protein, 493 aa, synthetic construct
  REGION 1..493: note = Formula III
  VARIANT 2: note = Any amino acid or absent
  VARIANT 6: note = Any amino acid or absent
  VARIANT 19: note = Any amino acid or absent
  VARIANT 22: note = Any amino acid or absent
  VARIANT 26: note = Any amino acid or absent
  VARIANT 46: note = Any amino acid or absent
  VARIANT 60: note = Any amino acid or absent
  VARIANT 72: note = Any amino acid or absent
  VARIANT 73: note = Any amino acid or absent
  VARIANT 174: note = Any amino acid or absent
  VARIANT 218: note = Any amino acid or absent
  VARIANT 221: note = Any amino acid or absent
  VARIANT 222: note = Any amino acid or absent
  VARIANT 224: note = Any amino acid or absent
  VARIANT 225: note = Any amino acid or absent
  VARIANT 226: note = Any amino acid or absent
  VARIANT 230: note = Any amino acid or absent
  VARIANT 235: note = Any amino acid or absent
  VARIANT 240: note = Any amino acid or absent
  VARIANT 242: note = Any amino acid or absent
  VARIANT 277: note = Any amino acid or absent
  VARIANT 281: note = Any amino acid or absent
  VARIANT 286: note = Any amino acid or absent
  VARIANT 287: note = Any amino acid or absent
  VARIANT 309: note = Any amino acid or absent
  VARIANT 312: note = Any amino acid or absent
  VARIANT 313: note = Any amino acid or absent
  VARIANT 316: note = Any amino acid or absent
  VARIANT 327: note = Any amino acid or absent
  VARIANT 328: note = Any amino acid or absent
  VARIANT 329: note = Any amino acid or absent
  VARIANT 382: note = Any amino acid or absent
  VARIANT 396: note = Any amino acid or absent
  VARIANT 397: note = Any amino acid or absent
  VARIANT 413: note = Any amino acid or absent
  VARIANT 414: note = Any amino acid or absent
  VARIANT 421: note = Any amino acid or absent
  VARIANT 425: note = Any amino acid or absent
  VARIANT 429: note = Any amino acid or absent
  VARIANT 432: note = Any amino acid or absent
  VARIANT 436: note = Any amino acid or absent
  VARIANT 441: note = Any amino acid or absent
  VARIANT 443: note = Any amino acid or absent
  VARIANT 444: note = Any amino acid or absent
  VARIANT 446: note = Any amino acid or absent
  VARIANT 466: note = Any amino acid or absent
  VARIANT 474: note = Any amino acid or absent
  VARIANT 477: note = Any amino acid or absent
  VARIANT 489: note = Any amino acid or absent
  VARIANT 492: note = Any amino acid or absent
  VARIANT 493: note = Any amino acid or absent
MXMDMXTRMFKEEGWIRKXCKXCGKXFWTLDPDRETCGDPPCDEYXFIGKPGIPKKYTLXEMREKFLSFFEXXGHGRVKRYPVLPRWRDDVLLVGASIMDFQPWVISGEADPPANPLTISQPSIRFTDIDNVGITGRHFTIFEMMAHHAFNYPGKPIYWMDETVELAFEFFTKXLGMKPEDITFKENPWAGGGNAGPAFEVLYRGLEVATLVFMQYKXAPXXAXXXQVVXIKGDXYVPMXTXVVDTGYGLERLVWMSQGTPTAYDAVLGYVVEPLKXMAGXEKIDXXILMENSRLAGMFDIEDMGDLRXLRXXVAXRVGISVEELEXXXRPYELIYAIADHTKALTFMLADGVIPSNVKAGYLARLLIRKSIRHLRELGLEXPLSEIVAMHIKELXXTFPEFKEMEDVILDIXXVEEKRYXETLXRGSXLVXREIXKLKKXGXXEXPLEKLILFYESHGLTPEIVXEIAEKEGXKVXIPDNFYSLVAKXAEXX

(19) United States
(12) Patent Application Publication
(10) Pub. No.: US 2026/0209742 A1
(43) Pub. Date: Jul. 23, 2026

(54) AMINOACYL-TRNA SYNTHETASES AND USES HEREOF

(71) Applicant: Novo Nordisk A/S, Bagsvaerd (DK)

(72) Inventor: Hongtao Guan, Beijing (CN)

(21) Appl. No.: 19/398,728

(22) Filed: Nov. 24, 2025

Related U.S. Application Data
(62) Division of application No. 17/431,452, filed on Feb. 19, 2020, now Pat. No. 12,509,675.

(30) Foreign Application Priority Data
Feb. 20, 2019 (WO) PCT/CN2019/075525
May 3, 2019 (EP) 19172611.6

(51) Int. Cl. C12N 9/00; C12P 21/02

(52) U.S. Cl. CPC C12N 9/93; C12P 21/02; C12Y 601/01

(57) ABSTRACT

The invention relates to aminoacyl-tRNA synthetases that aminoacylate tRNA with 2-Aminoisobutyric acid (Aib), thus enabling the incorporation of the Aib into a growing polypeptide chain during translation, e.g. in eubacterial host cells such as E. coli. For example, but not limited to, the invention relates to new aminoacyl-tRNA synthetases and the uses hereof, as well as a method for producing polypeptides that contain one or more Aib.

### CROSS-REFERENCE TO RELATED APPLICATIONS

This application is a divisional of U.S. application Ser. No. 17/431,452, filed Aug. 17, 2021, which is a 35 U.S.C. 371 national stage filing of International Application No. PCT/EP2020/054343, filed Feb. 19, 2020, which claims priority to EP Application No. 19172611.6, filed May 3, 2019 and which also claims priority to CN Application No. PCT/CN2019/075525, filed Feb. 20, 2019. The contents of the aforementioned applications are hereby incorporated by reference in their entireties.

### TECHNICAL FIELD

The invention is in the field of translation biochemistry. The invention relates to an aminoacyl-tRNA synthetase that can be used to incorporate unnatural amino acids into polypeptides.

### INCORPORATION-BY-REFERENCE OF THE SEQUENCE LISTING

The instant application contains a Sequence Listing which has been submitted in XML format via USPTO patent electronic filing system and is hereby incorporated by reference in its entirety. Said XML file, created on Nov. 21, 2025 and amended on Apr. 6, 2026, is named “180091 US02_Seq_Listing.xml”, and is 275,322 bytes in size.

### BACKGROUND

Recombinant expression is an efficient way to produce natural or engineered proteins and peptides (individually and collectively referred to as “polypeptides”). The vast majority of known organism encodes the same twenty naturally occurring amino acids, and consequently recombinant expression is, without further development, limited to polypeptides made exclusively from naturally occurring amino acids. One strategy to overcome this limitation has been developed for the in vivo site-specific incorporation of diverse unnatural amino acids into polypeptides in both prokaryotic and eukaryotic organisms (Wang L1, Schultz PG, A general approach for the generation of orthogonal tRNAs, Chem. Biol., 2001 Sep, 8(9):883-90; Liu CC, Schultz PG, Adding new chemistries to the genetic code, Annu. Rev. Biochem., 2010, 79:413-44.). These methods utilize an aminoacyl-tRNA synthetase (RS) that aminoacalytes a tRNA with a desired unnatural amino acid, which in turn incorporates that desired unnatural amino acid into a growing polypeptide chain in response to a selector codon during translation. The translation components may be developed to cross-react with the endogenous tRNAs, RSs, or amino acids in the host organism with reduced efficiency. 2-Aminoisobutyric acid (Aib) is a non-proteinogenic amino acid with the structural formula H₂N—C(CH₃)₂—COOH. Aib can be incorporated into a polypeptide and endow the polypeptide with desirable properties. One example of a polypeptide with desirable properties comprising Aib is semaglutide; a bioactive GLP-1 (glucagon-like peptide-1) analogue [Lau J. et. al, Discovery of the once-weekly glucagon-like peptide-1 (GLP-1) analogue semaglutide, J. Med. Chem., 2015; 58:7370-7380] that has been marketed as the antidiabetic drug Ozempic@. Semaglutide is disclosed in WO06097537.

No translation components have been reported to genetically encode Aib, and thus polypeptides containing Aib are currently precluded from being recombinantly expressed.

### SUMMARY

The invention relates to an aminoacyl-tRNA synthetase that aminoacylates tRNA with 2-Aminoisobutyric acid (Aib), thus enabling the incorporation of the Aib into a growing polypeptide chain during translation. In other words, the invention provides means for the preparation of polypeptides having translationally incorporated Aib.

In a first aspect, the invention relates to 2-Aminoisobutyric acid-tRNA synthetase (AibRS) that aminoacylates a tRNA with 2-Aminoisobutyric acid. In a second aspect, the invention relates to the use of AibRS for the preparation of polypeptides containing Aib. In a third aspect, invention relates to a method for preparing a compound comprising a polypeptide containing Aib, wherein the method comprises the step of using the AibRS for the preparation of the polypeptide containing Aib.

### DESCRIPTION

Unless otherwise indicated, in the specification and in the appended claims, terms presented in singular form also include the plural situation. Thus, the singular forms “a”, “an” and “the” include plural referents unless the content clearly dictates otherwise. E.g. reference to “a polypeptide” includes, as a practical matter, many polypeptides.

In a first aspect, the invention relates to 2-Aminoisobutyric acid-tRNA synthetase (AibRS). The 2-Aminoisobutyric acid-tRNA synthetase (AibRS) comprises an amino acid sequence of SEQ ID NO: 7 or a variant hereof, wherein the variant of SEQ ID NO: 7 has Gly in position 215.

In another aspect, the invention relates to the use of an AibRS of the invention for the preparation of a polypeptide containing Aib. In a further aspect, the invention relates to the use of an AibRS of the invention for the preparation of a resulting polypeptide comprising SEQ ID NO: 32.

In another aspect, the invention relates to a method for preparing Chem. 2 including the steps of (i) preparing a resulting polypeptide by using an AibRS of the invention and (ii) derivatisation of the resulting polypeptide.

### 2-Aminoisobutyric Acid (Aib)

The term “2-Aminoisobutyric acid “(Aib), as used herein, refers to the unnatural amino acid represented by the chemical structure Chem 1. Aib may also be referred to as “α-methylalanine”.

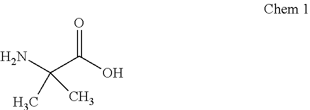

### 2-Aminoisobutyric Acid-tRNA Synthetase (AibRS)

As used herein, “2-Aminoisobutyric acid-tRNA synthetase” (AibRS) refers to an aminoacyl-tRNA synthetase (RS) that is capable of aminoacylating a tRNA with Aib. The AibRS may be: (i) identical or substantially similar to a naturally occurring alanine-tRNA synthetase (AlaRS), (ii) derived from a naturally occurring AlaRS by natural or artificial mutagenesis, (iii) or derived by any process that takes a sequence of a wild-type or mutant AlaRS sequence of (i) or (ii) into account, e.g. library screening or rational design. The AibRS may be derived from an AlaRS of Pyrococcus horikoshiior any naturally occurring AlaRS.

The term “translation”, as used herein, refers to a translation operation, or part of a translation operation, that is identical or similar to the translation operation of gene expression as known from the fields of molecular biology and genetics. The outcome of translation is a growing polypeptide chain which is also referred to as a “resulting polypeptide”. The AibRS of the invention may aminoacylate a tRNA with Aib making the tRNA available for translation, wherein the outcome of the translation is a resulting polypeptide containing one or more Aib residues. A tRNA that functions in this manner is also referred to as “tRNA^{Aib}”. In other words, the AibRS may be configured to incorporate Aib into a resulting polypeptide when used in connection with a translation process. The function of the Aib is not limited to any specific resulting polypeptide. The translation system is particularly suitable for preparing a resulting polypeptide comprising SEQ ID NO: 32.

The aminoacylatation of the tRNA with Aib by the AibRS may take place in competition with other amino acids, e.g. a natural amino acid, such as Ala, present in a host cell. In other words, the invention is not limited to an AibRS that exclusively functions to aminoacylate the tRNA of the translational system with Aib; the AibRS of the invention may aminoacylate the tRNA with any amino acid. Preferably, the AibRS aminoacylates the tRNA with Aib. The efficiency of the AibRS function may be determined by analysing the resulting polypeptide upon translation of the tRNA. The efficiency may be expressed as the ratio between the amount of resulting polypeptide containing Aib and the amount of resulting polypeptide containing another amino acid, e.g. Ala. The quantification of the resulting polypeptide may be carried out using LC-MS.

The function of the AibRS of the invention is not limited to any specific biological system. The AibRS may operate in context of in vitro conditions and/or in vivo conditions. The AibRS is suitable for operating in context of a host cell, where the translation of the resulting polypeptide containing Aib takes place by utilising one or more components of the translation machinery of the host cell. The AibRS may interact with endogenous components of a biological system in which the AibRS is utilised, e.g. components of a host cell.

The term “aminoacylation” as used herein, refers to the operation in which an AibRS (or any other aminoacyl-tRNA synthetase) catalyses the bonding between a tRNA and an amino acid. The AibRS is said to “aminoacylate” the tRNA. The term “aminoacylate” is used interchangeably with “charge”.

In one embodiment, the AibRS comprises an amino acid sequence of SEQ ID NO: 7 or a variant hereof, wherein the variant comprises 215Gly. In one embodiment the variant of SEQ ID NO: 7 is a variant of Formula I. In one embodiment, the variant of Formula I is at least 90% identical to SEQ ID NO: 7 in positions not designated Xaa. In one embodiment, the variant of Formula I is at least 50%, at least 60%, at least 70%, at least 75%, at least 80%, at least 85%, at least 90%, at least 95%, at least 99%, or at least 100% identical to Formula I in sequence positions that are not designated Xaa. In one embodiment, the variant of SEQ ID NO: 7 is at least 41%, at least 55%, or at least 90% identical to SEQ ID NO: 7. In one embodiment, the variant of SEQ ID NO: 7 is at least 50%, preferably at least 60%, more preferably at least 70%, more preferably at least 75%, more preferably at least 80%, more preferably at least 85%, more preferably at least 90%, more preferably at least 95%, and most preferably at least 99% identical to SEQ ID NO: 7. In one embodiment, the variant of SEQ ID NO: 7 is characterised in: position 192 is Trp, His, Val, lie, or Leu, position 193 is Ala, Leu, lie, or Gly, position 213 is Thr, Ser, Cys, or Ala, position 216 is Phe or Trp, position 217 is Met, lie, or Leu, position 249 is Thr, Ser, Val, or Phe, position 360 is Asn or Ala, and position 459 is Glu or Ala. In one embodiment, the variant of SEQ ID NO: 7 comprises: [192His; 215Gly]; [192His; 215Gly; 360Ala; 459Ala], [192His; 215Gly; 217Ile; 360Ala; 459Ala], [192His; 215Gly; 217Leu; 360Ala; 459Ala], [192His; 215Gly; 193Leu; 360Ala; 459Ala], [192His; 215Gly; 216Trp; 360Ala; 459Ala], [192His; 215Gly; 193Leu; 216Trp; 360Ala; 459Ala], [192His; 215Gly; 193Ile; 360Ala; 459Ala], [192His; 215Gly; 193Ile; 217Ile; 360Ala; 459Ala ], [192His; 215Gly; 193Leu; 217Ile; 360Ala; 459Ala ], [192His; 215Gly; 193Ile; 217Leu; 360Ala. 459Ala ], [192His; 215Gly; 193Leu; 217Leu; 360Ala; 459Ala ], [192Val; 215Gly; 217Ile; 360Ala; 459Ala ], [192Ile; 215Gly; 217Ile; 360Ala; 459Ala], [192Leu; 215Gly; 217Ile; 360Ala; 459Ala], [192His; 193Gly; 215Gly; 217Ile; 360Ala; 459Ala ], [192His; 213Ser; 215Gly; 360Ala; 459Ala ], [192His; 215Gly; 249Ser; 360Ala; 459Ala], [192His; 215Gly; 249Val; 360Ala; 459Ala], [192His; 213Cys; 215Gly; 249Val; 360Ala; 459Ala], or [192His; 213Ala; 215Gly; 249Phe; 360Ala; 459Ala]. In one embodiment, the variant of SEQ ID NO: 7 is selected from a list consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30. In one embodiment, the AibRS is derived from an archaeal bacterium. In one embodiment, the AibRS is derived from Pyrococcus horikoshii. In one embodiment, the AibRS is configured to perform the function of aminoacylating a tRNA with Aib. In one embodiment, the AibRS is capable of aminoacylating a tRNA with Aib. In one embodiment, the AibRS is configured to perform the function of aminoacylating a tRNA with Aib in a host cell. In one embodiment, the AibRS is configured to perform the function of aminoacylating a tRNA with Aib in E. coli. In one embodiment, the efficiency of the function of aminoacylating a tRNA with Aib is determined by analysing the expression product, i.e. the resulting polypeptide upon translation of the tRNA. In one embodiment, the efficiency is expressed as the incorporation ratio between the resulting polypeptide containing Aib and the amount of resulting polypeptide containing Ala or Aib in the position intended for Aib. In one embodiment, the incorporation ratio is determined using LC-MS and calculated based on the mass spectrum as follows: Incorporation ratio=[Peak intensity]_{Aib-containing polypeptide}/([Peak intensity]_{Aib-containing polypeptide}+[Peak intensity]Ala-containing polypeptide)*100%. In one embodiment, the incorporation ratio is at least 1%, at least 5%, at least 10%, at least 15%, at least 20%, at least 25%, at least 30%, at least 35%, at least 40%, at least 45%, at least 50%, at least 55, at least 60%, at least 65%, or at least 70%.

tRNA^{Aib}

As used herein, “tRNA^{Aib}” refers to a tRNA which can be aminoacylated with Aib by AibRS, and which incorporates Aib into a resulting polypeptide during translation. The tRNA may be: (i) identical or substantially similar to a naturally occurring alanine-tRNA (tRNA^{Ala}) (ii) derived from a naturally occurring tRNA^{Ala} by natural or artificial mutagenesis, (iii) derived by any process that takes a sequence of a wild-type or mutant tRNA^{Ala} sequence of (1) or (2) into account, e.g. library screening and/or rational design. In some embodiments, the tRNA may be derived from Pyrococcus horikoshii. The tRNA^{Aib} may be a suppressor tRNA.

The tRNA^{Aib} can exist in a charged stated (i.e. aminoacylated with an amino acid), or in an uncharged state (i.e. not aminoacylated with an amino acid). The tRNA^{Aib} is cognate to the AibRS of the invention and is aminoacylated with Aib. The aminoacylatation of the tRNA by the AibRS with Aib may take place in competition with another amino acid, e.g. a natural amino acid, such as Ala, present in a host cell. In other words, the tRNA^{Aib} is not limited to a tRNA molecule that is aminoacylated exclusively with Aib by the AibRS, the tRNA^{Aib} may be aminoacylated with another amino acid by the AibRS. The tRNA^{Aib} functions to insert an amino acid into a resulting polypeptide during translation in response to a selector codon. Preferably, that amino acid is Aib.

The term “in response to”, as used herein, refers to the process in which a tRNA of the invention recognizes a selector codon and mediates the incorporation of the unnatural amino acid, which is coupled to the tRNA, into a resulting polypeptide. The tRNA^{Aib} may incorporate Aib in response to a selector codon that is a stop codon, e.g. TAG.

The term “encode”, as used herein, refers to any process whereby the information in a molecule or sequence string is used to direct the production of a second molecule or sequence string that is different from the first molecule or sequence string. In particular, an RNA molecule can encode a polypeptide, in which case a translation process is required to take place before the resulting polypeptide can be obtained. When used to describe the process of translation, the term “encode” may also extend to the triplet codon that encodes an amino acid or a stop codon. In particular a DNA molecule can encode a resulting polypeptide, in which case both a transcription process and a translation process are required to take place before the resulting peptide can be obtained. It is to be understood that the present invention is not limited to work in context of polynucleotides, for which only a translation process is required to generate the resulting polypeptide (e.g. RNA). The invention may also work in context of polynucleotides, for which both a transcription process and a translation process are required to generate the resulting polypeptide (e.g. DNA).

In one embodiment, the AibRS is configured to perform the function of aminoacylating a tRNA with Aib. In one embodiment, the tRNA is a suppressor tRNA. In one embodiment, the tRNA comprises one or more anticodons that encodes Aib. In one embodiment, the anticodon of the tRNA that encodes Aib is complementary to a stop codon. In one embodiment, the selector codon is a nonsense codon, e.g. a stop codon, a four-base codon, a rare codon, and a codon derived from natural, unnatural base pairs and/or the like. In one embodiment, the stop codon is an amber, an ochre, and/or an opal codon. In one embodiment, the anticodon that encodes Aib is CTA. In one embodiment, the tRNA is encoded by SEQ ID NO: 3 or a variant hereof. In one embodiment, the variant of SEQ ID NO: 3 encoding the tRNA contains a G3A mutation. In one embodiment, the variant of SEQ ID NO: 3 encoding the tRNA is at least 50%, at least 60%, at least 70% at least 75%, at least 80%, at least 85%, at least 90%, at least 95%, at least 99%, or at least 100% identical to SEQ ID NO: 3. In one embodiment, the variant of SEQ ID NO: 3 encoding the tRNA is SEQ ID NO: 4. In one embodiment, the tRNA is encoded by SEQ ID NO: 4. In one embodiment, the tRNA is derived from an archaeal bacterium. In one embodiment, the tRNA is derived from Pyrococcus horikoshii.

### Amino Acids

The term “natural amino acid”, as used herein, refers the 20 standard amino acids which are encoded by the standard genetic code in humans. Natural amino acids may also be referred to as “proteinogenic amino acids”. The term “unnatural amino acid”, refers to amino acids that may exist in nature (but not fall under the 20 standard amino acids which are encoded by the standard genetic code in humans), or be purely synthetic, e.g. modified amino acids and amino acids analogues. Unnatural amino acids may also be referred to as “non-proteinogenic amino acids” or “non-coded amino acids”. Non-limiting examples of unnatural amino acids are Aib and D-isomers of the natural amino acids. If the term “amino acid” is used herein without indication of whether or not it is natural or unnatural, it is to be construed as if it includes both natural and unnatural amino acids. The term “any amino acid” as used herein is to be construed as if it includes both natural and unnatural amino acids.

Suppressor tRNA and Selector Codons

The term “suppressor tRNA” as used herein, refers to a tRNA that alters the reading of an mRNA in a given translation system, allowing for translational read-through of a codon (e.g. a selector codon that is a stop codon) that would otherwise result in the termination of translation or mistranslation (e.g., frame-shifting). Typically, the suppressor tRNA allows for the incorporation of an amino acid in response to a stop codon during the translation of a polypeptide (a process that is referred to as “read-through”).

The term “selector codon”, as used herein, refers to a codon in response to which the tRNA (e.g. suppressor tRNA) incorporates an amino acid into the resulting polypeptide. The tRNA is said to be an “anticodon” to the selector codon. The selector codon may be a nonsense codon, e.g. a stop codon, a four-base codon, a rare codon, and a codon derived from natural, unnatural base pairs and/or the like. Non-limiting examples of stop codons are amber, ochre, and opal codons.

### Mutations and Mutants

The term “mutation”, as used herein in context of an amino acid sequence or a polypeptide, refers to an amino acid (i) being substituted by another amino acid, (ii) being deleted, or (iii) being added. Mutations in context of an amino acid sequence may be referred to as “amino acid changes”. A specific amino acid in a specific position of an amino acid sequence (or a polypeptide) may be described by reference to the sequence position in question followed by a reference to the three-letter code of the amino acid that is present in that position. A non-limiting example of this nomenclature in context of an amino acid sequence or polypeptide is: “1 92His and 215Gly”, meaning that His is present in position 192 and Gly is present in position 215. Substitutions in an amino acid sequence (or a polypeptide) may be described by reference to the three-letter code of the amino acid that is substituted, followed by reference to the sequence position in question, followed by a reference to the amino acid that is substituted with. A non-limiting example of this nomenclature is: “Trp192His; Val215Gly”, wherein Trp at position 192 has been substituted with His, and Val at position 215 has been substituted with Gly. Substitutions may be provided in relation to a specific sequence described in the sequence listing (typically in relation to a wild-type tRNA synthetase), and the position of the amino acid that is substituted is as such determined based on a numbering that can be derived from that particular sequence in the sequence listing. In the sequence listing, the first amino acid residue (counting from the N-terminus) of SEQ ID NO: 1 (Met) is assigned no. 1; the second amino acid residue of SEQ ID NO: 1 (Glu) is assigned no. 2 and so forth. The term “mutation”, and as used herein in context of a nucleotide sequence or a polynucleotide, refers to a nucleotide (i) being substituted by another nucleotide, (ii) being deleted, or (iii) being added.

Substitutions in nucleotide sequences or polynucleotides may be described by reference to the one-letter code of the nucleobase of the nucleotide that is substituted, followed by reference to the sequence position of the nucleotide that is substituted, followed by a reference to the nucleobase of the nucleotide that is substituted with. A non-limiting example of substitution nomenclature in context of a nucleotide sequence or a polynucleotide is: “G3A”, wherein the guanine has been replaced with the adenine. Substitutions may be provided in relation to a specific sequence described in the sequence listing (typically in relation to wild-type tRNA), and the position of the nucleotide that is substituted is as such determined based on a numbering that can be derived from that particular sequence in the sequence listing. In the sequence listing, the first nucleotide residue (counting from the 5′-end) of SEQ ID NO: 2 (G) is assigned no. 1; the second amino acid residue of SEQ ID NO: 2 (G) is assigned no. 2 and so forth.

The term “mutant”, as used herein in context of an amino acid sequence or a polypeptide refers to a sequence that has one or more mutations as compared to the sequence it is a mutant of.

### Variant

The term “variant”, as used herein, refers to a component that may have structural differences as compared to the component that it is a variant of, but it maintains a similar functionality. E.g. an AibRS and a variant hereof do not necessarily share the same amino acid sequence, but they will both aminoacylate a cognate tRNA with an unnatural amino acid. A variant of an AibRS of the invention may have one or more mutations, as compared to the AibRS, as long as the variant still functions to aminoacylate a tRNA with Aib. Mutations may include substitutions, deletions and additions. The mutations in a variant may also be referred to as “variations”.

### Polynucleotides and Polypeptides

The term “polynucleotide”, as used herein, refers to a sequence of two or more nucleotides. The terms “polynucleotide” and “nucleotide sequence” are used interchangeably. The term “polypeptide”, as used herein, refers to a sequence of two or more, natural or unnatural, amino acids. As such, the term “polypeptide” encompasses both peptides and proteins. The terms “polypeptide” and “amino acid sequence” are used interchangeably. The term “resulting polypeptide”, as used herein, refers to a polypeptide that is the product of a translation process, typically involving the AibRS of the invention. The resulting polypeptide may also be referred to as a “growing polypeptide chain”, especially when the resulting polypeptide is described in context of the translation process. The AibRS of the invention is particularly suitable for the preparation of the resulting polypeptide comprising SEQ ID NO: 32, which is the amino acid sequence of semaglutide [Lau J. et. al, Discovery of the once-weekly glucagon-like peptide-1 (GLP-1) analogue semaglutide, J. Med. Chem., 2015; 58:7370-7380].

In one embodiment, the AibRS is used for the preparation of a resulting polypeptide containing one or more Aib residues. In one embodiment, the AibRS is used for the preparation of a resulting polypeptide containing one or more Aib residues, wherein the resulting polypeptide containing one or more Aib residues is a GLP-1 analogue. In one embodiment, the AibRS is used for the preparation of a resulting polypeptide, wherein the resulting polypeptide comprises SEQ ID NO: 32. In one embodiment, the AibRS is used for the preparation of a resulting polypeptide, wherein the resulting polypeptide is SEQ ID NO: 32.

### Derivative

The term “derivative”, as used herein, in the context of a polypeptide means a chemically modified polypeptide, in which one or more substituents have been covalently attached to the polypeptide. The term “derivatisation”, as used herein, refers to the process of obtaining the derivative from the polypeptide. E.g. Chem. 2 may be obtained from SEQ ID NO: 32 by derivatisation introducing a substituent (may also be referred to as “side chain”) of Chem. 3.

The AibRS of the invention is particularly useful in a method of preparing semaglutide (Chem. 2) comprising the steps of (i) preparation of a resulting polypeptide comprising SEQ ID NO: 32 by using the AibRS of the invention and (ii) derivatisation of the polypeptide comprising SEQ ID NO: 32 with the substituent Chem. 3.

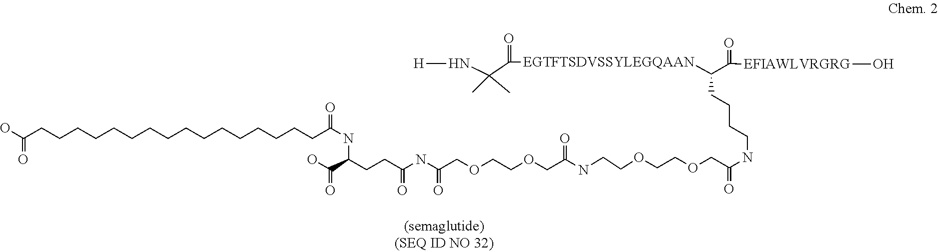

Chem. 3 (substituent [or side chain] of semaglutide; wherein * indicates the point of attachment to the polypeptide sequence):

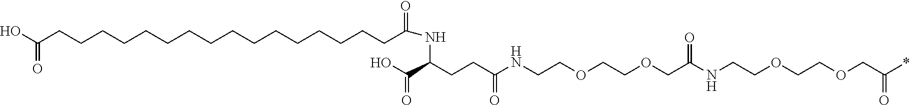 [SMILES: *C(=O)COCCOCCNC(=O)COCCOCCNC(=O)CC[C@H](NC(=O)CCCCCCCCCCCCCCCCC(=O)O)C(=O)O]

In one embodiment, the invention relates to a method of producing a compound comprising the steps of: (i) preparation of a resulting polypeptide containing one or more Aib residues using the AibRS of the invention, and (ii) derivatisation of the resulting polypeptide; in a preferred embodiment the compound is Chem. 2; in a preferred embodiment, the resulting polypeptide is SEQ ID NO: 32; in a preferred embodiment, and the resulting polypeptide is derivatised with Chem. 3. Derivatisation may be carried out as described in Lau J. et. al, Discovery of the once-weekly glucagon-like peptide-1 (GLP-1) analogue semaglutide, J. Med. Chem., 2015; 58:7370-7380.

### Cognate

As used herein, “cognate” refers to components that function together, or have some aspect of specificity for each other, e.g. a tRNA^{Aib} and an AibRS, as well as an anticodon and a stop codon. The components can also be referred to as being “complementary”. In one embodiment the AibRS is cognate to a tRNA^{Aib}.

### Orthogonal

The term “orthogonal” as used herein refers to a molecule (e.g., an AibRS and/or a tRNA^{Aib}) that functions with endogenous components of a host cell with reduced efficiency as compared to a corresponding molecule that is endogenous to the host cell or that fails to function with endogenous components of the cell. In the context of tRNAs and RS, orthogonal refers to an inability or reduced efficiency of an orthogonal tRNA to function with an endogenous RS compared to an endogenous tRNA to function with the endogenous RS, or of an orthogonal RS to function with an endogenous tRNA compared to an endogenous RS to function with the endogenous tRNA. The orthogonal molecule lacks a functionally normal endogenous complementary molecule in the cell. For example, an orthogonal tRNA in a cell is aminoacylated by any endogenous RS of the cell with reduced or even zero efficiency, when compared to aminoacylation of an endogenous tRNA by the endogenous RS. In another example, an orthogonal RS aminoacylates any endogenous tRNA a cell of interest with reduced or even zero efficiency, as compared to aminoacylation of the endogenous tRNA by an endogenous RS. Orthogonality may be expressed as the efficiency of the AibRS function.

The translational components (e.g. an AibRS and/or a tRNA^{Aib}) of the invention may be derived from any organism (or a combination of organisms) for use in a host translation system from any other species, with the caveat that the translational components and the host system function in an orthogonal manner. In some embodiments, the translational components are derived from Archaea genes (i.e. archaebacteria) for use in a eubacterial host system. For example, the orthogonal tRNA^{Aib} can be derived from an archae organism, e. g. an archaebacterium, such as Methanococcus jannaschii, Methanobacterium thermoautotrophicum, Halobacterium (e.g. Haloferax volcanii and Halobacterium species NRC-I), Archaeoglobus fulgidus, Pyrococcus furiosus, Pyrococcus horikoshii, Aeuropyrum pernix, Methanococcus maripaludis, Methanopyrus kandleri, Methanosarcina mazei, Pyrobaculum aerophilum, Pyrococcus abyssi, Sulfolobus solfataricus, Sulfolobus lokodaili, Thermoplasma acidophilum, Thermoplasma volcanium, or the like, or a eubaclerium, such as E. coli, and Thermus thermophilus. In some embodiments, the translational components are derived from eukaryotic sources, e.g., plants, algae, protists, fungi, yeasts, animals (e.g., mammals, insects, arthropods, etc.), or the like.

### Host Cell

The term “host cell”, as used herein refers to a cell in which the AibRS of the invention may carry out its function. The host cell may be prokaryotic, e.g. bacteria and archaea, or eukaryotic, e.g. yeast, algae, filamentous fungus, a mammalian cell, a plant cell, and an insect cell. A microbial host cell may also be referred to as a “microorganism”. Non-limiting examples of eubacteria include E. coli, Thermus thermophilus, Bacillus subtilis, Bacillus stearothermophilus, Corynebacterium glutamicum. Non-limiting examples of archaea include Methanococcus jannaschii, Methanosarcina mazei, Methanobacterium thermoautotrophicum, Methanococcus maripaludis, Methanopyrus kandleri, Halobacterium (e.g. Haloferax volcanii and Halobacterium species NRC-I), Archaeoglobus fulgidus, Pyrococcus furiosus, Pyrococcus horikoshii, Pyrobaculum aerophilum, Pyrococcus abyssi, Sulfolobus solfataricus, Sulfolobus tokodaii, Aeuropyrum pernix, Thermoplasma acidophilum and Thermoplasma volcanium. Non-limiting examples of yeast and filamentous fungi include Saccharomyces cerevisiae, Pichia pastoris, Pichia pfaffi, Hansenula polymorpha, Aspergillus niger and Trichoderma resei. Non-limiting examples of mammalian cells include CHO, CHO-K1, CHO-DXB11, CHO-DG44, CHO-S, HEK293 or derivatives of any of these cells.

Host cells are typically genetically engineered (e.g., transformed, transduced or transfected) with the polynucleotide encoding the AibRS of the invention, e.g. using one or more vectors. The coding regions for the AibRS of the invention, and the polypeptide to be translated may be operably linked to gene expression control elements that are functional in the desired host cell. Vectors may contain transcription and translation terminators, transcription and translation initiation sequences, as well as promoters useful for regulation of the expression of the particular target nucleic acid. The vectors may comprise generic expression cassettes containing at least one independent terminator sequence, sequences permitting replication of the cassette in eukaryotes, or prokaryotes, or both (e.g., shuttle vectors) and selection markers for both prokaryotic and eukaryotic systems. Non-limiting examples of vectors are a plasmid, a bacterium, a virus, a naked polynucleotide, or a conjugated polynucleotide. In one embodiment, the host cell is E. coli.

### Consensus Sequence

The term “consensus sequence”, as used herein, refers to the calculated order of most frequent amino acid residues found at each position in a sequence alignment. It represents the results of multiple sequence alignments in which related sequences (e.g. Archaean AlaRS sequences) are compared to each other and similar sequence motifs are calculated. Thus, a consensus sequence is a model for the putative amino acid positions essential for functionality (e.g. by being involved in folding and/or amino acid binding) of the bioactive polypeptide. In general, there is a high degree of freedom for amino acids sequence positions not identified as a putative site of activity since the amino acids of these positions are often solvent-exposed in loops, and therefore a high degree of variation can be introduced in these positions while maintaining the function exercised by the bioactive polypeptide. In one embodiment, the Aib is defined as a variant of a consensus sequence. In one embodiment the Aib is defined as a variant of Formula I. In one embodiment the Aib is defined as a variant of Formula II. In one embodiment the Aib is defined as a variant of Formula III.

### Sequence Identity

Sequence identity is the extent to which two (nucleotide or amino acid) sequences have the same residues at the same positions in an alignment. The sequence identity is conveniently expressed as a percentage, i.e. if 85 amino acids out of 100 aligned positions between the two sequences are identical the degree of identity is 85%. If one of the two sequences is a consensus sequence, then only the conserved positions of the consensus sequence is considered for the calculation. I.e. if 85 amino acids out of 100 aligned and conserved positions are identical, then the degree of identity is 85% even though the sequences may be longer than 100 amino acids. For purposes of the present invention, the sequence identity between two amino acid sequences is determined by using simple handwriting and eyeballing; and/or a standard protein or peptide alignment program, such as “align” which is based on a Needleman-Wunsch algorithm. This algorithm is described in Needleman, S. B. and Wunsch, C. D., (1970), Journal of Molecular Biology, 48: 443-453, and the align program by Myers and W. Miller in “Optimal Alignments in Linear Space” CABIOS (computer applications in the biosciences) (1988) 4:11-17. For the alignment, the default scoring matrix BLOSUM62 and the default identity matrix may be used, and the penalty for the first residue in a gap may be set at −12, or preferably at −10, and the penalties for additional residues in a gap at −2, or preferably at −0.5.

### Particular Embodiments

It is to be understood that the terminology used herein is for the purpose of describing particular embodiments, and thus it is not intended to be limiting.
1. A 2-Aminoisobutyric acid-tRNA synthetase (AibRS) comprising an amino acid sequence of SEQ ID NO: 7 or a variant hereof, wherein the variant comprises 215Gly.
2. The AibRS according to any preceding embodiment; wherein the variant of SEQ ID NO: 7 is a variant of Formula I; wherein Formula I is:

(SEQ ID NO: 33)
Xaa-Xaa-Xaa-Xaa-Xaa-Xaa-Xaa-Xaa-Xaa-Phe-Xaa-Glu-
Xaa-Gly-Xaa-Xaa-Xaa-Lys-Xaa-Cys-Xaa-Xaa-Cys-Gly-
Xaa-Xaa-Phe-Trp-Thr-Xaa-Xaa-Xaa-Xaa-Arg-Xaa-Xaa-
Cys-Gly-Asp-Xaa-Pro-Cys-Xaa-Xaa-Tyr-Xaa-Phe-Ile-
Gly-Xaa-Pro-Xaa-Xaa-Xaa-Xaa-Xaa-Xaa-Xaa-Leu-Xaa-
Glu-Xaa-Arg-Xaa-Xaa-Phe-Xaa-Xaa-Phe-Phe-Glu-Xaa-
Xaa-Xaa-His-Xaa-Xaa-Xaa-Xaa-Arg-Tyr-Pro-Val-Xaa-
Xaa-Arg-Trp-Arg-Asp-Asp-Val-Xaa-Leu-Val-Gly-Ala-
Ser-Ile-Xaa-Asp-Phe-Gln-Pro-Trp-Val-Xaa-Xaa-Gly-
Xaa-Xaa-Xaa-Pro-Pro-Ala-Asn-Pro-Leu-Xaa-Ile-Ser-
Gln-Pro-Xaa-Ile-Arg-Xaa-Xaa-Asp-Xaa-Asp-Xaa-Val-
Gly-Xaa-Xaa-Gly-Arg-His-Xaa-Thr-Xaa-Phe-Glu-Met-
Met-Ala-His-His-Ala-Phe-Asn-Xaa-Xaa-Xaa-Xaa-Xaa-
Xaa-Tyr-Trp-Xaa-Xaa-Glu-Thr-Val-Xaa-Xaa-Xaa-Xaa-
Xaa-Phe-Xaa-Xaa-Xaa-Xaa-Xaa-Xaa-Xaa-Xaa-Xaa-Xaa-
Xaa-Ile-Thr-Phe-Xaa-Glu-Xaa-Xaa-Trp-Xaa-Gly-Gly-
Gly-Asn-Ala-Gly-Xaa-Xaa-Xaa-Glu-Val-Xaa-Xaa-Xaa-
Gly-Xaa-Glu-Xaa-Ala-Thr-Leu-Val-Phe-Met-Xaa-Tyr-
Xaa-Xaa-Xaa-Xaa-Xaa-Xaa-Xaa-Xaa-Xaa-Xaa-Xaa-Xaa-
Xaa-Xaa-Xaa-Xaa-Xaa-Xaa-Xaa-Xaa-Xaa-Pro-Xaa-Xaa-
Xaa-Xaa-Xaa-Val-Asp-Thr-Gly-Tyr-Gly-Leu-Glu-Arg-
Xaa-Xaa-Trp-Xaa-Ser-Xaa-Gly-Xaa-Pro-Thr-Xaa-Tyr-
Asp-Ala-Xaa-Xaa-Xaa-Xaa-Xaa-Xaa-Xaa-Xaa-Leu-Xaa-
Xaa-Xaa-Ala-Gly-Xaa-Xaa-Xaa-Xaa-Xaa-Xaa-Xaa-Xaa-
Ile-Leu-Xaa-Xaa-Xaa-Xaa-Xaa-Xaa-Ala-Gly-Xaa-Xaa-
Asp-Xaa-Xaa-Xaa-Xaa-Xaa-Xaa-Leu-Xaa-Xaa-Leu-Arg-
Xaa-Xaa-Val-Ala-Xaa-Xaa-Xaa-Xaa-Xaa-Xaa-Xaa-Xaa-
Glu-Leu-Xaa-Xaa-Xaa-Xaa-Xaa-Pro-Xaa-Glu-Xaa-Xaa-
Tyr-Ala-Ile-Ala-Asp-His-Thr-Xaa-Xaa-Leu-Xaa-Phe-
Met-Leu-Xaa-Asp-Gly-Val-Xaa-Pro-Ser-Asn-Xaa-Xaa-
Ala-Gly-Tyr-Leu-Ala-Arg-Leu-Xaa-Ile-Arg-Xaa-Xaa-
Xaa-Arg-Xaa-Xaa-Xaa-Xaa-Leu-Gly-Xaa-Xaa-Xaa-Pro-
Leu-Xaa-Xaa-Ile-Xaa-Xaa-Xaa-Xaa-Xaa-Xaa-Xaa-Xaa-
Xaa-Xaa-Xaa-Xaa-Pro-Glu-Xaa-Xaa-Glu-Xaa-Xaa-Xaa-
Xaa-Xaa-Xaa-Xaa-Xaa-Xaa-Xaa-Xaa-Glu-Glu-Xaa-Xaa-
Xaa-Xaa-Xaa-Thr-Xaa-Xaa-Arg-Gly-Xaa-Xaa-Xaa-Xaa-
Xaa-Xaa-Xaa-Xaa-Xaa-Xaa-Xaa-Lys-Xaa-Xaa-Gly-Xaa-
Xaa-Xaa-Xaa-Pro-Xaa-Xaa-Xaa-Leu-Xaa-Xaa-Xaa-Tyr-
Xaa-Ser-His-Gly-Xaa-Xaa-Pro-Glu-Xaa-Xaa-Xaa-Glu-
Xaa-Ala-Xaa-Xaa-Xaa-Xaa-Xaa-Xaa-Val-Xaa-Xaa-Pro-
Asp-Asn-Phe-Tyr-Xaa-Xaa-Val-Ala-Xaa-Xaa-Xaa-Xaa-
Xaa-Xaa

wherein each Xaa in Formula I is independently selected and is one or more amino acids, or absent;
wherein the variant of Formula I is at least 90% identical to SEQ ID NO: 7 in positions not designated Xaa.
3. The AibRS according to any preceding embodiment; wherein the variant of SEQ ID NO: 7 is a variant of Formula I; wherein Formula I corresponds to a consensus sequence of the following formula:

(SEQ ID NO: 33)
Xaa-Xaa-Xaa-Xaa-Xaa-Xaa-Xaa-Xaa-Xaa-Phe-Xaa-Glu-
Xaa-Gly-Xaa-Xaa-Xaa-Lys-Xaa-Cys-Xaa-Xaa-Cys-Gly-
Xaa-Xaa-Phe-Trp-Thr-Xaa-Xaa-Xaa-Xaa-Arg-Xaa-Xaa-
Cys-Gly-Asp-Xaa-Pro-Cys-Xaa-Xaa-Tyr-Xaa-Phe-Ile-
Gly-Xaa-Pro-Xaa-Xaa-Xaa-Xaa-Xaa-Xaa-Xaa-Leu-Xaa-
Glu-Xaa-Arg-Xaa-Xaa-Phe-Xaa-Xaa-Phe-Phe-Glu-Xaa-
Xaa-Xaa-His-Xaa-Xaa-Xaa-Xaa-Arg-Tyr-Pro-Val-Xaa-
Xaa-Arg-Trp-Arg-Asp-Asp-Val-Xaa-Leu-Val-Gly-Ala-
Ser-Ile-Xaa-Asp-Phe-Gln-Pro-Trp-Val-Xaa-Xaa-Gly-
Xaa-Xaa-Xaa-Pro-Pro-Ala-Asn-Pro-Leu-Xaa-Ile-Ser-
Gln-Pro-Xaa-Ile-Arg-Xaa-Xaa-Asp-Xaa-Asp-Xaa-Val-
Gly-Xaa-Xaa-Gly-Arg-His-Xaa-Thr-Xaa-Phe-Glu-Met-
Met-Ala-His-His-Ala-Phe-Asn-Xaa-Xaa-Xaa-Xaa-Xaa-
Xaa-Tyr-Trp-Xaa-Xaa-Glu-Thr-Val-Xaa-Xaa-Xaa-Xaa-
Xaa-Phe-Xaa-Xaa-Xaa-Xaa-Xaa-Xaa-Xaa-Xaa-Xaa-Xaa-
Xaa-Ile-Thr-Phe-Xaa-Glu-Xaa-Xaa-Trp-Xaa-Gly-Gly-
Gly-Asn-Ala-Gly-Xaa-Xaa-Xaa-Glu-Val-Xaa-Xaa-Xaa-
Gly-Xaa-Glu-Xaa-Ala-Thr-Leu-Val-Phe-Met-Xaa-Tyr-
Xaa-Xaa-Xaa-Xaa-Xaa-Xaa-Xaa-Xaa-Xaa-Xaa-Xaa-Xaa-
Xaa-Xaa-Xaa-Xaa-Xaa-Xaa-Xaa-Xaa-Xaa-Pro-Xaa-Xaa-
Xaa-Xaa-Xaa-Val-Asp-Thr-Gly-Tyr-Gly-Leu-Glu-Arg-
Xaa-Xaa-Trp-Xaa-Ser-Xaa-Gly-Xaa-Pro-Thr-Xaa-Tyr-
Asp-Ala-Xaa-Xaa-Xaa-Xaa-Xaa-Xaa-Xaa-Xaa-Leu-Xaa-
Xaa-Xaa-Ala-Gly-Xaa-Xaa-Xaa-Xaa-Xaa-Xaa-Xaa-Xaa-
Ile-Leu-Xaa-Xaa-Xaa-Xaa-Xaa-Xaa-Ala-Gly-Xaa-Xaa-
Asp-Xaa-Xaa-Xaa-Xaa-Xaa-Xaa-Leu-Xaa-Xaa-Leu-Arg-
Xaa-Xaa-Val-Ala-Xaa-Xaa-Xaa-Xaa-Xaa-Xaa-Xaa-Xaa-
Glu-Leu-Xaa-Xaa-Xaa-Xaa-Xaa-Pro-Xaa-Glu-Xaa-Xaa-
Tyr-Ala-Ile-Ala-Asp-His-Thr-Xaa-Xaa-Leu-Xaa-Phe-
Met-Leu-Xaa-Asp-Gly-Val-Xaa-Pro-Ser-Asn-Xaa-Xaa-
Ala-Gly-Tyr-Leu-Ala-Arg-Leu-Xaa-Ile-Arg-Xaa-Xaa-
Xaa-Arg-Xaa-Xaa-Xaa-Xaa-Leu-Gly-Xaa-Xaa-Xaa-Pro-
Leu-Xaa-Xaa-Ile-Xaa-Xaa-Xaa-Xaa-Xaa-Xaa-Xaa-Xaa-
Xaa-Xaa-Xaa-Xaa-Pro-Glu-Xaa-Xaa-Glu-Xaa-Xaa-Xaa-
Xaa-Xaa-Xaa-Xaa-Xaa-Xaa-Xaa-Xaa-Glu-Glu-Xaa-Xaa-
Xaa-Xaa-Xaa-Thr-Xaa-Xaa-Arg-Gly-Xaa-Xaa-Xaa-Xaa-
Xaa-Xaa-Xaa-Xaa-Xaa-Xaa-Xaa-Lys-Xaa-Xaa-Gly-Xaa-
Xaa-Xaa-Xaa-Pro-Xaa-Xaa-Xaa-Leu-Xaa-Xaa-Xaa-Tyr-
Xaa-Ser-His-Gly-Xaa-Xaa-Pro-Glu-Xaa-Xaa-Xaa-Glu-
Xaa-Ala-Xaa-Xaa-Xaa-Xaa-Xaa-Xaa-Val-Xaa-Xaa-Pro-
Asp-Asn-Phe-Tyr-Xaa-Xaa-Val-Ala-Xaa-Xaa-Xaa-Xaa-
Xaa-Xaa

wherein each Xaa of Formula I is independently selected and is one or more amino acids, or absent;
wherein the variant of Formula I is at least 90% identical to SEQ ID NO: 7 in positions not designated Xaa.
4. The AibRS according to any preceding embodiment; wherein the variant of Formula I is at least 50%, at least 60%, at least 70%, at least 75%, at least 80%, at least 85%, at least 90%, at least 95%, at least 99%, or at least 100% identical to Formula I in sequence positions that are not designated Xaa.
5. The AibRS according to any preceding embodiment, wherein Xaa is one or more amino acids of any kind, or absent.
6. The AibRS according to any preceding embodiment, wherein the Xaa of Formula I is any amino acid or absent.
7. The AibRS according to any preceding embodiment; wherein the Xaa of Formula I is any amino acid.
8. The AibRS according to any preceding embodiment, wherein the variant of SEQ ID NO: 7 is at least 41% identical to SEQ ID NO: 7.
9. The AibRS according to any preceding embodiment, wherein the variant of SEQ ID NO: 7 is at least 55% identical to SEQ ID NO: 7.
10. The AibRS according to any preceding embodiment, wherein the variant of SEQ ID NO: 7 is at least 90% identical to SEQ ID NO: 7.
11. The AibRS according to any preceding embodiment, wherein the variant of SEQ ID NO: 7 is at least 50%, preferably at least 60%, more preferably at least 70%, more preferably at least 75%, more preferably at least 80%, more preferably at least 85%, more preferably at least 90%, more preferably at least 95%, and most preferably at least 99% identical to SEQ ID NO: 7.
12. The AibRS according to any preceding embodiment; wherein the variant of SEQ ID NO: 7 is characterised in:
   position 192 is Trp, His, Val, lie, or Leu
   position 193 is Ala, Leu, lie, or Gly
   position 213 is Thr, Ser, Cys, or Ala
   position 216 is Phe or Trp
   position 217 is Met, lie, or Leu
   position 249 is Thr, Ser, Val, or Phe
   position 360 is Asn or Ala,
   position 459 is Glu or Ala.
13. The AibRS according to any preceding embodiment; wherein the variant of SEQ ID NO: 7 is characterised in:
   position 192 is His, Val, lie, or Leu
   position 193 is Ala, Leu, lie, or Gly
   position 213 is Thr, Ser, Cys, or Ala
   position 216 is Phe or Trp
   position 217 is Met, lie, or Leu
   position 249 is Thr, Ser, Val, or Phe
   position 360 is Ala,
   position 459 is Ala.
14. The AibRS according to any preceding embodiment; wherein the variant of SEQ ID NO: 7 comprises: [192His; 215Gly]; [192His; 215Gly; 360Ala; 459Ala], [192His; 215Gly; 217Ile; 360Ala; 459AIa], [192His; 215Gly; 217Leu; 360Ala; 459AIa], [192His; 215Gly; 193Leu; 360Ala; 459AIa], [192His; 215Gly; 216Trp; 360Ala; 459AIa], [192His; 215Gly; 193Leu; 216Trp; 360Ala; 459AIa], [192His; 215Gly; 193Ile; 360Ala; 459AIa], [192His; 215Gly; 193Ile; 217Ile; 360Ala; 459Ala ], [192His; 215Gly; 193Leu; 217Ile; 360Ala; 459Ala ], [192His; 215Gly; 193Ile; 217Leu; 360Ala. 459Ala ], [192His; 215Gly; 193Leu; 217Leu; 360Ala; 459AIa ], [192Val; 215Gly; 217Ile; 360Ala; 459AIa ], [192Ile; 215Gly; 217Ile; 360Ala; 459AIa], [192Leu; 215Gly; 217Ile; 360Ala; 459AIa], [192His; 193Gly; 215Gly; 217Ile; 360Ala; 459AIa ], [192His; 213Ser; 215Gly; 360Ala; 459AIa ], [192His; 215Gly; 249Ser; 360Ala; 459AIa], [192His; 215Gly; 249Val; 360Ala; 459AIa], [192His; 213Cys; 215Gly; 249Val; 360Ala; 459AIa], or [192His; 213Ala; 215Gly; 249Phe; 360Ala; 459AIa].
15. The AibRS according to any preceding embodiment; wherein the variant of SEQ ID NO: 7 comprises: [192His; 215Gly; 360Ala; 459AIa], [192His; 215Gly; 217Ile; 360Ala; 459AIa], [192His; 215Gly; 217Leu; 360Ala; 459AIa], [192His; 215Gly; 193Leu; 360Ala; 459AIa], [192His; 215Gly; 216Trp; 360Ala; 459AIa], [192His; 215Gly; 193Leu; 216Trp; 360Ala; 459AIa], [192His; 215Gly; 193Ile; 360Ala; 459AIa], [192His; 215Gly; 193Ile; 217Ile; 360Ala; 459AIa ], [192His; 215Gly; 193Leu; 217Ile; 360Ala; 459AIa ], [192His; 215Gly; 193Ile; 217Leu; 360Ala. 459AIa ], [192His; 215Gly; 193Leu; 217Leu; 360Ala; 459Ala ], [192Val; 215Gly; 217Ile; 360Ala; 459Ala ], [192Ile; 215Gly; 217Ile; 360Ala; 459Ala], [192Leu; 215Gly; 217Ile; 360Ala; 459Ala], [192His; 193Gly; 215Gly; 217Ile; 360Ala; 459Ala ], [192His; 213Ser; 215Gly; 360Ala; 459Ala ], [192His; 215Gly; 249Ser; 360Ala; 459Ala], [192His; 215Gly; 249Val; 360Ala; 459Ala], [192His; 213Cys; 215Gly; 249Val; 360Ala; 459Ala], or [192His; 213Ala; 215Gly; 249Phe; 360Ala; 459Ala].
16. The AibRS according to any preceding embodiment; wherein the variant of SEQ ID NO: 7 is selected from a list consisting of SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.
17. The AibRS according to any preceding embodiment; wherein the variant of SEQ ID NO: 7 is selected from a list consisting of SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.
18. The AibRS according to any preceding embodiment; wherein the AibRS is derived from an archaeal bacterium.
19. The AibRS according to any preceding embodiment; wherein the AibRS is derived from Pyrococcus horikoshii.
20. The AibRS according to any preceding embodiment; wherein the AibRS is configured to perform the function of aminoacylating a tRNA with Aib.
21. The AibRS according to any preceding claim; wherein the AibRS is capable of aminoacylating a tRNA with Aib.
22. The AibRS according to any preceding embodiment; wherein the tRNA is a suppressor tRNA.
23. The AibRS according to any preceding embodiment; wherein the tRNA comprises one or more anticodons that encodes Aib.
24. The AibRS according to any preceding embodiment; wherein the anticodon of the tRNA that encodes Aib is complementary to a stop codon.
25. The AibRS according to any preceding embodiment; wherein the anticodon that encodes Aib is CTA.
26. The AibRS according to any preceding embodiment; wherein the tRNA is encoded by SEQ ID NO: 3 or a variant hereof.
27. The AibRS according to any preceding embodiment; wherein the variant of SEQ ID NO: 3 encoding the tRNA contains a G3A mutation.
28. The AibRS according to any preceding embodiment; wherein the variant of SEQ ID NO: 3 encoding the tRNA is at least 50%, at least 60%, at least 70% at least 75%, at least 80%, at least 85%, at least 90%, at least 95%, at least 99%, or at least 100% identical to SEQ ID NO: 3.
29. The AibRS according to any preceding embodiment; wherein the variant of SEQ ID NO: 3 encoding the tRNA is SEQ ID NO: 4.
30. The AibRS according to any preceding embodiment; wherein the tRNA is encoded by SEQ ID NO: 4.
31. The AibRS according to any preceding embodiment; wherein the tRNA is derived from an archaeal bacterium.
32. The AibRS according to any preceding embodiment; wherein the tRNA is derived from Pyrococcus horikoshii.
33. The AibRS according to any preceding embodiment; wherein the AibRS is configured to perform the function of aminoacylating a tRNA with Aib in a host cell.
34. The AibRS according to any preceding embodiment; wherein the AibRS is configured to perform the function of aminoacylating a tRNA with Aib in E. coli.
35. The AibRS according to any preceding embodiment; wherein the efficiency of the function is determined by analysing the expression product, i.e. the resulting polypeptide upon translation of the tRNA.
36. The AibRS according to any preceding embodiment; wherein the efficiency is expressed as the incorporation ratio between the resulting polypeptide containing Aib and the amount of resulting polypeptide containing Ala or Aib in the position intended for Aib.
37. The AibRS according to any preceding embodiment; wherein the incorporation ratio is determined using LC-MS and calculated based on the mass spectrum as follows:

$$Incorporation ratio = {[ Peak intensity ]}_{Aib - containing polypeptide} / ( {[ Peak intensity ]}_{Aib - containing polypeptide} + {[ Peak intensity ]}_{Ala - containing polypeptide} ) * 100 \% .$$

38. The AibRS according to any preceding embodiment; wherein the incorporation ratio is at least 1%, at least 5%, at least 10%, at least 15%, at least 20%, at least 25%, at least 30%, at least 35%, at least 40%, at least 45%, at least 50%, at least 55, at least 60%, at least 65%, or at least 70%.
39. The use of an AibRS according to any preceding embodiment for the preparation of a resulting polypeptide containing one or more Aib residues.
40. The use of an AibRS, according to any preceding embodiment, wherein the resulting polypeptide containing one or more Aib residues is a GLP-1 analogue.
41. The use of an AibRS according to any preceding embodiment, wherein the resulting polypeptide containing one or more Aib residues comprises SEQ ID NO: 32.
42. A method of producing a compound comprising the steps of:
   i. preparation of a resulting polypeptide containing one or more Aib residues using the AibRS of any preceding embodiment,
   ii. derivatisation of the resulting polypeptide
43. A method according to any of the preceding embodiments; wherein the compound is

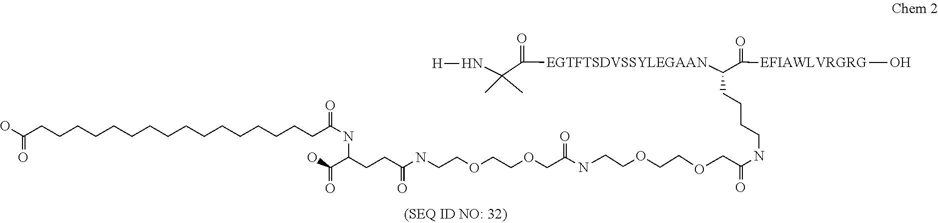

### Further Particular Embodiments

It is to be understood that the terminology used herein is for the purpose of describing particular embodiments, and thus it is not intended to be limiting.
1. A 2-Aminoisobutyric acid-tRNA synthetase (AibRS) comprising an amino acid sequence of SEQ ID NO: 7 or a variant hereof, wherein the variant comprises 215Gly.
2. An AibRS comprising an amino acid sequence of SEQ ID NO: 7, or a variant hereof, wherein the variant is at least 41%, at least 50%, at least 55%, at least 60%, at least 70%, at least 80%, at least 90%, at least 95%, at least 99%, or at least 100% identical to SEQ ID NO: 7.
3. An AibRS comprising an amino acid sequence of SEQ ID NO: 10, or a variant hereof, wherein the variant is at least 41%, at least 50%, at least 55%, at least 60% at least 70%, at least 80%, at least 90%, at least 95%, at least 99%, or at least 100% identical to SEQ ID NO: 10.
4. An AibRS comprising an amino acid sequence of SEQ ID NO: 11, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 11.
5. An AibRS comprising an amino acid sequence of SEQ ID NO: 12, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 12.
6. An AibRS comprising an amino acid sequence of SEQ ID NO: 13, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 13.
7. An AibRS comprising an amino acid sequence of SEQ ID NO: 14, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 14.
8. An AibRS comprising an amino acid sequence of SEQ ID NO: 15, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 15.
9. An AibRS comprising an amino acid sequence of SEQ ID NO: 16, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 16.
10. An AibRS comprising an amino acid sequence of SEQ ID NO: 17, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 17.
11. An AibRS comprising an amino acid sequence of SEQ ID NO: 18, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 18.
12. An AibRS comprising an amino acid sequence of SEQ ID NO: 19, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 19.
13. An AibRS comprising an amino acid sequence of SEQ ID NO: 20, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 20.
14. An AibRS comprising an amino acid sequence of SEQ ID NO: 21, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 21.
15. An AibRS comprising an amino acid sequence of SEQ ID NO: 22, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 22.
16. An AibRS comprising an amino acid sequence of SEQ ID NO: 23, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 23.
17. An AibRS comprising an amino acid sequence of SEQ ID NO: 24, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 24.
18. An AibRS comprising an amino acid sequence of SEQ ID NO: 25, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 25.
19. An AibRS comprising an amino acid sequence of SEQ ID NO: 26, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 26.
20. An AibRS comprising an amino acid sequence of SEQ ID NO: 27, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 27.
21. An AibRS comprising an amino acid sequence of SEQ ID NO: 28, or a variant hereof, wherein the variant of variant is at least 41%, at least 50%, at least 55%, at least 60% at least 70%, at least 80%, at least 90%, at least 95%, at least 99%, or at least 100% identical to SEQ ID NO: 28.
22. An AibRS comprising an amino acid sequence of SEQ ID NO: 29, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 29.
23. An AibRS comprising an amino acid sequence of SEQ ID NO: 30, or a variant hereof, wherein the variant variant is at least 41%, at least 50%, at least 55%, at least 60% at least 70%, at least 80%, at least 90%, at least 95%, at least 99%, or at least 100% identical to SEQ ID NO: 30.
24. The AibRS according to any of the embodiments 4-23, wherein the variant comprises 215Gly.
25. The AibRS according to any preceding embodiment; wherein the variant is of Formula I or a variant hereof:

Formula I:
(SEQ ID NO: 33)
Xaa-Xaa-Xaa-Xaa-Xaa-Xaa-Xaa-Xaa-Xaa-Phe-Xaa-Glu-
Xaa-Gly-Xaa-Xaa-Xaa-Lys-Xaa-Cys-Xaa-Xaa-Cys-Gly-
Xaa-Xaa-Phe-Trp-Thr-Xaa-Xaa-Xaa-Xaa-Arg-Xaa-Xaa-
Cys-Gly-Asp-Xaa-Pro-Cys-Xaa-Xaa-Tyr-Xaa-Phe-Ile-
Gly-Xaa-Pro-Xaa-Xaa-Xaa-Xaa-Xaa-Xaa-Xaa-Leu-Xaa-
Glu-Xaa-Arg-Xaa-Xaa-Phe-Xaa-Xaa-Phe-Phe-Glu-Xaa-
Xaa-Xaa-His-Xaa-Xaa-Xaa-Xaa-Arg-Tyr-Pro-Val-Xaa-
Xaa-Arg-Trp-Arg-Asp-Asp-Val-Xaa-Leu-Val-Gly-Ala-
Ser-Ile-Xaa-Asp-Phe-Gln-Pro-Trp-Val-Xaa-Xaa-Gly-
Xaa-Xaa-Xaa-Pro-Pro-Ala-Asn-Pro-Leu-Xaa-Ile-Ser-
Gln-Pro-Xaa-Ile-Arg-Xaa-Xaa-Asp-Xaa-Asp-Xaa-Val-
Gly-Xaa-Xaa-Gly-Arg-His-Xaa-Thr-Xaa-Phe-Glu-Met-
Met-Ala-His-His-Ala-Phe-Asn-Xaa-Xaa-Xaa-Xaa-Xaa-
Xaa-Tyr-Trp-Xaa-Xaa-Glu-Thr-Val-Xaa-Xaa-Xaa-Xaa-
Xaa-Phe-Xaa-Xaa-Xaa-Xaa-Xaa-Xaa-Xaa-Xaa-Xaa-Xaa-
Xaa-Ile-Thr-Phe-Xaa-Glu-Xaa-Xaa-Trp-Xaa-Gly-Gly-
Gly-Asn-Ala-Gly-Xaa-Xaa-Xaa-Glu-Val-Xaa-Xaa-Xaa-
Gly-Xaa-Glu-Xaa-Ala-Thr-Leu-Val-Phe-Met-Xaa-Tyr-
Xaa-Xaa-Xaa-Xaa-Xaa-Xaa-Xaa-Xaa-Xaa-Xaa-Xaa-Xaa-
Xaa-Xaa-Xaa-Xaa-Xaa-Xaa-Xaa-Xaa-Xaa-Pro-Xaa-Xaa-
Xaa-Xaa-Xaa-Val-Asp-Thr-Gly-Tyr-Gly-Leu-Glu-Arg-
Xaa-Xaa-Trp-Xaa-Ser-Xaa-Gly-Xaa-Pro-Thr-Xaa-Tyr-
Asp-Ala-Xaa-Xaa-Xaa-Xaa-Xaa-Xaa-Xaa-Xaa-Leu-Xaa-
Xaa-Xaa-Ala-Gly-Xaa-Xaa-Xaa-Xaa-Xaa-Xaa-Xaa-Xaa-
Ile-Leu-Xaa-Xaa-Xaa-Xaa-Xaa-Xaa-Ala-Gly-Xaa-Xaa-
Asp-Xaa-Xaa-Xaa-Xaa-Xaa-Xaa-Leu-Xaa-Xaa-Leu-Arg-
Xaa-Xaa-Val-Ala-Xaa-Xaa-Xaa-Xaa-Xaa-Xaa-Xaa-Xaa-
Glu-Leu-Xaa-Xaa-Xaa-Xaa-Xaa-Pro-Xaa-Glu-Xaa-Xaa-
Tyr-Ala-Ile-Ala-Asp-His-Thr-Xaa-Xaa-Leu-Xaa-Phe-
Met-Leu-Xaa-Asp-Gly-Val-Xaa-Pro-Ser-Asn-Xaa-Xaa-
Ala-Gly-Tyr-Leu-Ala-Arg-Leu-Xaa-Ile-Arg-Xaa-Xaa-
Xaa-Arg-Xaa-Xaa-Xaa-Xaa-Leu-Gly-Xaa-Xaa-Xaa-Pro-
Leu-Xaa-Xaa-Ile-Xaa-Xaa-Xaa-Xaa-Xaa-Xaa-Xaa-Xaa-
Xaa-Xaa-Xaa-Xaa-Pro-Glu-Xaa-Xaa-Glu-Xaa-Xaa-Xaa-
Xaa-Xaa-Xaa-Xaa-Xaa-Xaa-Xaa-Xaa-Glu-Glu-Xaa-Xaa-
Xaa-Xaa-Xaa-Thr-Xaa-Xaa-Arg-Gly-Xaa-Xaa-Xaa-Xaa-
Xaa-Xaa-Xaa-Xaa-Xaa-Xaa-Xaa-Lys-Xaa-Xaa-Gly-Xaa-
Xaa-Xaa-Xaa-Pro-Xaa-Xaa-Xaa-Leu-Xaa-Xaa-Xaa-Tyr-
Xaa-Ser-His-Gly-Xaa-Xaa-Pro-Glu-Xaa-Xaa-Xaa-Glu-
Xaa-Ala-Xaa-Xaa-Xaa-Xaa-Xaa-Xaa-Val-Xaa-Xaa-Pro-
Asp-Asn-Phe-Tyr-Xaa-Xaa-Val-Ala-Xaa-Xaa-Xaa-Xaa-
Xaa-Xaa,

wherein Xaa is one or more amino acids of any kind, or absent.
26. The AibRS according to any preceding embodiment; wherein the variant of Formula I is at least 50%, at least 60%, at least 70%, at least 75%, at least 80%, at least 85%, at least 90%, at least 95%, at least 99%, or at least 100% identical to Formula I in sequence positions that are not designated Xaa.
27. The AibRS according to any preceding embodiment; wherein the Xaa of Formula I is any amino acid or absent.
28. The AibRS according to any preceding embodiment; wherein the Xaa of Formula I is any amino acid.
29. The AibRS according to any preceding embodiment; wherein the variant is of Formula II or a variant hereof:

Formula II:
(SEQ ID NO: 34)
Xaa-Xaa-Xaa-Xaa-Xaa-Xaa-Xaa-Xaa-Xaa-Phe-Xaa-Glu-
Xaa-Gly-Xaa-Xaa-Arg-Lys-Xaa-Cys-Xaa-Xaa-Cys-Gly-
Xaa-Xaa-Phe-Trp-Thr-Xaa-Asp-Pro-Asp-Arg-Glu-Thr-
Cys-Gly-Asp-Xaa-Pro-Cys-Asp-Xaa-Tyr-Xaa-Phe-Ile-
Gly-Xaa-Pro-Xaa-Xaa-Xaa-Xaa-Xaa-Tyr-Xaa-Leu-Xaa-
Glu-Xaa-Arg-Glu-Xaa-Phe-Leu-Xaa-Phe-Phe-Glu-Xaa-
Xaa-Xaa-His-Xaa-Arg-Xaa-Xaa-Arg-Tyr-Pro-Val-Xaa-
Xaa-Arg-Trp-Arg-Asp-Asp-Val-Xaa-Leu-Val-Gly-Ala-
Ser-Ile-Xaa-Asp-Phe-Gln-Pro-Trp-Val-Xaa-Ser-Gly-
Xaa-Xaa-Xaa-Pro-Pro-Ala-Asn-Pro-Leu-Xaa-Ile-Ser-
Gln-Pro-Ser-Ile-Arg-Xaa-Thr-Asp-Xaa-Asp-Asn-Val-
Gly-Xaa-Thr-Gly-Arg-His-Xaa-Thr-Xaa-Phe-Glu-Met-
Met-Ala-His-His-Ala-Phe-Asn-Xaa-Pro-Xaa-Xaa-Xaa-
Xaa-Tyr-Trp-Xaa-Asp-Glu-Thr-Val-Glu-Xaa-Xaa-Xaa-
Xaa-Phe-Xaa-Thr-Xaa-Xaa-Leu-Xaa-Xaa-Xaa-Xaa-Glu-
Xaa-Ile-Thr-Phe-Lys-Glu-Xaa-Xaa-Trp-Xaa-Gly-Gly-
Gly-Asn-Ala-Gly-Pro-Xaa-Xaa-Glu-Val-Leu-Xaa-Arg-
Gly-Leu-Glu-Val-Ala-Thr-Leu-Val-Phe-Met-Gln-Tyr-
Lys-Xaa-Xaa-Xaa-Xaa-Xaa-Xaa-Xaa-Xaa-Xaa-Xaa-Xaa-
Xaa-Xaa-Xaa-Xaa-Gly-Xaa-Xaa-Xaa-Xaa-Pro-Met-Xaa-
Xaa-Xaa-Xaa-Val-Asp-Thr-Gly-Tyr-Gly-Leu-Glu-Arg-
Xaa-Val-Trp-Xaa-Ser-Xaa-Gly-Thr-Pro-Thr-Ala-Tyr-
Asp-Ala-Val-Xaa-Xaa-Xaa-Val-Xaa-Xaa-Xaa-Leu-Xaa-
Xaa-Xaa-Ala-Gly-Xaa-Xaa-Xaa-Xaa-Xaa-Xaa-Xaa-Xaa-
Ile-Leu-Xaa-Glu-Asn-Ser-Xaa-Leu-Ala-Gly-Xaa-Xaa-
Asp-Ile-Glu-Xaa-Xaa-Xaa-Asp-Leu-Xaa-Xaa-Leu-Arg-
Xaa-Xaa-Val-Ala-Xaa-Xaa-Xaa-Gly-Ile-Xaa-Xaa-Xaa-
Glu-Leu-Xaa-Xaa-Xaa-Xaa-Xaa-Pro-Xaa-Glu-Xaa-Ile-
Tyr-Ala-Ile-Ala-Asp-His-Thr-Xaa-Xaa-Leu-Xaa-Phe-
Met-Leu-Xaa-Asp-Gly-Val-Xaa-Pro-Ser-Asn-Val-Lys-
Ala-Gly-Tyr-Leu-Ala-Arg-Leu-Leu-Ile-Arg-Xaa-Xaa-
Xaa-Arg-Xaa-Xaa-Xaa-Xaa-Leu-Gly-Leu-Xaa-Xaa-Pro-
Leu-Xaa-Glu-Ile-Xaa-Xaa-Xaa-Xaa-Xaa-Xaa-Xaa-Leu-
Xaa-Xaa-Xaa-Xaa-Pro-Glu-Xaa-Xaa-Glu-Xaa-Xaa-Xaa-
Xaa-Ile-Xaa-Xaa-Xaa-Xaa-Xaa-Xaa-Glu-Glu-Xaa-Xaa-
Tyr-Xaa-Xaa-Thr-Leu-Xaa-Arg-Gly-Xaa-Xaa-Leu-Val-
Xaa-Xaa-Xaa-Xaa-Xaa-Xaa-Leu-Lys-Lys-Xaa-Gly-Xaa-
Xaa-Glu-Xaa-Pro-Leu-Glu-Xaa-Leu-Xaa-Xaa-Xaa-Tyr-
Xaa-Ser-His-Gly-Xaa-Xaa-Pro-Glu-Xaa-Val-Xaa-Glu-
Xaa-Ala-Xaa-Xaa-Xaa-Gly-Xaa-Xaa-Val-Xaa-Xaa-Pro-
Asp-Asn-Phe-Tyr-Xaa-Leu-Val-Ala-Xaa-Xaa-Xaa-Glu-
Xaa-Xaa,

wherein Xaa is one or more amino acids of any kind, or absent.
30. The AibRS according to any preceding embodiment; wherein the variant of Formula II is at least 50%, at least 60%, at least 70%, at least 75%, at least 80%, at least 85%, at least 90%, at least 95%, at least 99%, or at least 100% identical to Formula II in sequence positions that are not designated Xaa.
31. The AibRS according to any preceding embodiment; wherein the Xaa of Formula II is any amino acid or absent.
32. The AibRS according to any preceding embodiment; wherein the Xaa of Formula II is any amino acid.
33. The AibRS according to any preceding embodiment; wherein the variant is of Formula III or a variant hereof:

Formula III:
(SEQ ID NO: 35)
Met-Xaa-Met-Asp-Met-Xaa-Thr-Arg-Met-Phe-Lys-Glu-
Glu-Gly-Trp-Ile-Arg-Lys-Xaa-Cys-Lys-Xaa-Cys-Gly-
Lys-Xaa-Phe-Trp-Thr-Leu-Asp-Pro-Asp-Arg-Glu-Thr-
Cys-Gly-Asp-Pro-Pro-Cys-Asp-Glu-Tyr-Xaa-Phe-Ile-
Gly-Lys-Pro-Gly-Ile-Pro-Lys-Lys-Tyr-Thr-Leu-Xaa-
Glu-Met-Arg-Glu-Lys-Phe-Leu-Ser-Phe-Phe-Glu-Xaa-
Xaa-Gly-His-Gly-Arg-Val-Lys-Arg-Tyr-Pro-Val-Leu-
Pro-Arg-Trp-Arg-Asp-Asp-Val-Leu-Leu-Val-Gly-Ala-
Ser-Ile-Met-Asp-Phe-Gln-Pro-Trp-Val-Ile-Ser-Gly-
Glu-Ala-Asp-Pro-Pro-Ala-Asn-Pro-Leu-Thr-Ile-Ser-
Gln-Pro-Ser-Ile-Arg-Phe-Thr-Asp-Ile-Asp-Asn-Val-
Gly-Ile-Thr-Gly-Arg-His-Phe-Thr-Ile-Phe-Glu-Met-
Met-Ala-His-His-Ala-Phe-Asn-Tyr-Pro-Gly-Lys-Pro-
Ile-Tyr-Trp-Met-Asp-Glu-Thr-Val-Glu-Leu-Ala-Phe-
Glu-Phe-Phe-Thr-Lys-Xaa-Leu-Gly-Met-Lys-Pro-Glu-
Asp-Ile-Thr-Phe-Lys-Glu-Asn-Pro-Trp-Ala-Gly-Gly-
Gly-Asn-Ala-Gly-Pro-Ala-Phe-Glu-Val-Leu-Tyr-Arg-
Gly-Leu-Glu-Val-Ala-Thr-Leu-Val-Phe-Met-Gln-Tyr-
Lys-Xaa-Ala-Pro-Xaa-Xaa-Ala-Xaa-Xaa-Xaa-Gln-Val-
Val-Xaa-Ile-Lys-Gly-Asp-Xaa-Tyr-Val-Pro-Met-Xaa-
Thr-Xaa-Val-Val-Asp-Thr-Gly-Tyr-Gly-Leu-Glu-Arg-
Leu-Val-Trp-Met-Ser-Gln-Gly-Thr-Pro-Thr-Ala-Tyr-
Asp-Ala-Val-Leu-Gly-Tyr-Val-Val-Glu-Pro-Leu-Lys-
Xaa-Met-Ala-Gly-Xaa-Glu-Lys-Ile-Asp-Xaa-Xaa-Ile-
Leu-Met-Glu-Asn-Ser-Arg-Leu-Ala-Gly-Met-Phe-Asp-
Ile-Glu-Asp-Met-Gly-Asp-Leu-Arg-Xaa-Leu-Arg-Xaa-
Xaa-Val-Ala-Xaa-Arg-Val-Gly-Ile-Ser-Val-Glu-Glu-
Leu-Glu-Xaa-Xaa-Xaa-Arg-Pro-Tyr-Glu-Leu-Ile-Tyr-
Ala-Ile-Ala-Asp-His-Thr-Lys-Ala-Leu-Thr-Phe-Met-
Leu-Ala-Asp-Gly-Val-Ile-Pro-Ser-Asn-Val-Lys-Ala-
Gly-Tyr-Leu-Ala-Arg-Leu-Leu-Ile-Arg-Lys-Ser-Ile-
Arg-His-Leu-Arg-Glu-Leu-Gly-Leu-Glu-Xaa-Pro-Leu-
Ser-Glu-Ile-Val-Ala-Met-His-Ile-Lys-Glu-Leu-Xaa-
Xaa-Thr-Phe-Pro-Glu-Phe-Lys-Glu-Met-Glu-Asp-Val-
Ile-Leu-Asp-Ile-Xaa-Xaa-Val-Glu-Glu-Lys-Arg-Tyr-
Xaa-Glu-Thr-Leu-Xaa-Arg-Gly-Ser-Xaa-Leu-Val-Xaa-
Arg-Glu-Ile-Xaa-Lys-Leu-Lys-Lys-Xaa-Gly-Xaa-Xaa-
Glu-Xaa-Pro-Leu-Glu-Lys-Leu-Ile-Leu-Phe-Tyr-Glu-
Ser-His-Gly-Leu-Thr-Pro-Glu-Ile-Val-Xaa-Glu-Ile-
Ala-Glu-Lys-Glu-Gly-Xaa-Lys-Val-Xaa-Ile-Pro-Asp-
Asn-Phe-Tyr-Ser-Leu-Val-Ala-Lys-Xaa-Ala-Glu-Xaa-
Xaa,

wherein Xaa is one or more amino acids of any kind, or absent.
34. The AibRS according to any preceding embodiment; wherein the variant of Formula III is at least 50%, at least 60%, at least 70%, at least 75%, at least 80%, at least 85%, at least 90%, at least 95%, at least 99%, or at least 100% identical to Formula III in sequence positions that are not designated Xaa.
35. The AibRS according to any preceding embodiment; wherein the Xaa of Formula III is any amino acid or absent.
36. The AibRS according to any preceding embodiment; wherein the Xaa of Formula III is any amino acid.
37. The AibRS according to any preceding embodiment; wherein the variant comprises at least 197 amino acids selected from a list consisting of: 10Phe, 12Glu, 14Gly, 18Lys, 20Cys, 23Cys, 24Gly, 27Phe, 28Trp, 29Thr, 34Arg, 37Cys, 38Gly, 39Asp, 41 Pro, 42Cys, 45Tyr, 47Phe, 48Ile, 49Gly, 51 Pro, 59Leu, 61Glu, 63Arg, 66Phe, 69Phe, 70Phe, 71Glu, 75His, 80Arg, 81Tyr, 82Pro, 83Val, 86Arg, 87Trp, 88Arg, 89Asp, 90Asp, 91Val, 93Leu, 94Val, 95Gly, 96Ala, 97Ser, 98Ile, 100Asp, 101 Phe, 102Gln, 103Pro, 104Trp, 105Val, 108Gly, 112Pro, 113Pro, 114Ala, 115Asn, 116Pro, 117Leu, 119Ile, 120Ser, 121Gln, 122Pro, 124Ile, 125Arg, 128Asp, 130Asp, 132Val, 133Gly, 136Gly, 137Arg, 138His, 140Thr, 142Phe, 143Glu, 144Met, 145Met, 146Ala, 147His, 148His, 149Ala, 150Phe, 151Asn, 158Tyr, 159Trp, 162Glu, 163Thr, 164Val, 170Phe, 182Ile, 183Thr, 184Phe, 186Glu, 189Trp, 191Gly, 192Gly, 193Gly, 194Asn, 195Ala, 196Gly, 200Glu, 201Val, 205Gly, 207Glu, 209Ala, 210Thr, 211Leu, 212Val, 213Phe, 214Met, 216Tyr, 238Pro, 244Val, 245Asp, 246Thr, 247Gly, 248Tyr, 249Gly, 250Leu, 251 Glu, 252Arg, 255Trp, 257Ser, 259Gly, 261 Pro, 262Thr, 264Tyr, 265Asp, 266Ala, 275Leu, 279Ala, 280Gly, 289Ile, 290Leu, 297Ala, 298Gly, 301Asp, 308Leu, 311 Leu, 312Arg, 315Val, 316Ala, 325Glu, 326Leu, 332Pro, 334Glu, 337Tyr, 338Ala, 339Ile, 340Ala, 341Asp, 342His, 343Thr, 346Leu, 348Phe, 349Met, 350Leu, 352Asp, 353Gly, 354Val, 356Pro, 357Ser, 358Asn, 361Ala, 362Gly, 363Tyr, 364Leu, 365Ala, 366Arg, 367Leu, 369Ile, 370Arg, 374Arg, 379Leu, 380Gly, 384Pro, 385Leu, 388Ile, 401 Pro, 402Glu, 405Glu, 417Glu, 418Glu, 424Thr, 427Arg, 428Gly, 440Lys, 443Gly, 448Pro, 452Leu, 456Tyr, 458Ser, 459His, 460Gly, 463Pro, 464Glu, 468Glu, 470Ala, 477Val, 480Pro, 481Asp, 482Asn, 483Phe, 484Tyr, 487Val, and 488Ala;
38. The AibRS according to any preceding embodiment; wherein the variant comprises at least 264 amino acids selected from a list consisting of: 10Phe, 12Glu, 14Gly, 17Arg, 18Lys, 20Cys, 23Cys, 24Gly, 27Phe, 28Trp, 29Thr, 31Asp, 32Pro, 33Asp, 34Arg, 35Glu, 36Thr, 37Cys, 38Gly, 39Asp, 41 Pro, 42Cys, 43Asp, 45Tyr, 47Phe, 48Ile, 49Gly, 51 Pro, 57Tyr, 59Leu, 61Glu, 63Arg, 64Glu, 66Phe, 67Leu, 69Phe, 70Phe, 71Glu, 75His, 77Arg, 80Arg, 81Tyr, 82Pro, 83Val, 86Arg, 87Trp, 88Arg, 89Asp, 90Asp, 91Val, 93Leu, 94Val, 95Gly, 96Ala, 97Ser, 98Ile, 100Asp, 101Phe, 102GIn, 103Pro, 104Trp, 105Val, 107Ser, 108Gly, 112Pro, 113Pro, 114Ala, 115Asn, 116Pro, 117Leu, 119Ile, 120Ser, 121Gln, 122Pro, 123Ser, 124Ile, 125Arg, 127Thr, 128Asp, 130Asp, 131Asn, 132Val, 133Gly, 135Thr, 136Gly, 137Arg, 138His, 140Thr, 142Phe, 143Glu, 144Met, 145Met, 146Ala, 147His, 148His, 149Ala, 150Phe, 151Asn, 153Pro, 158Tyr, 159Trp, 161Asp, 162Glu, 163Thr, 164Val, 165Glu, 170Phe, 172Thr, 175Leu, 180Glu, 182Ile, 183Thr, 184Phe, 185Lys, 186Glu, 189Trp, 191Gly, 192Gly, 193Gly, 194Asn, 195Ala, 196Gly, 197Pro, 200Glu, 201Val, 202Leu, 204Arg, 205Gly, 206Leu, 207Glu, 208Val, 209Ala, 210Thr, 211Leu, 212Val, 213Phe, 214Met, 215GIn, 216Tyr, 217Lys, 233Gly, 238Pro, 239Met, 244Val, 245Asp, 246Thr, 247Gly, 248Tyr, 249Gly, 250Leu, 251Glu, 252Arg, 254Val, 255Trp, 257Ser, 259Gly, 260Thr, 261 Pro, 262Thr, 263Ala, 264Tyr, 265Asp, 266Ala, 267Val, 271Val, 275Leu, 279Ala, 280Gly, 289Ile, 290Leu, 292Glu, 293Asn, 294Ser, 296Leu, 297Ala, 298Gly, 301Asp, 302Ile, 303Glu, 307Asp, 308Leu, 311 Leu, 312Arg, 315Val, 316Ala, 320Gly, 321Ile, 325Glu, 326Leu, 332Pro, 334Glu, 336Ile, 337Tyr, 338Ala, 339Ile, 340Ala, 341Asp, 342His, 343Thr, 346Leu, 348Phe, 349Met, 350Leu, 352Asp, 353Gly, 354Val, 356Pro, 357Ser, 358Asn, 359Val, 360Lys, 361Ala, 362Gly, 363Tyr, 364Leu, 365Ala, 366Arg, 367Leu, 368Leu, 369Ile, 370Arg, 374Arg, 379Leu, 380Gly, 381 Leu, 384Pro, 385Leu, 387Glu, 388Ile, 396Leu, 401 Pro, 402Glu, 405Glu, 410Ile, 417Glu, 418Glu, 421Tyr, 424Thr, 425Leu, 427Arg, 428Gly, 431Leu, 432Val, 439Leu, 440Lys, 441 Lys, 443Gly, 446Glu, 448Pro, 449Leu, 450Glu, 452Leu, 456Tyr, 458Ser, 459His, 460Gly, 463Pro, 464Glu, 466Val, 468Glu, 470Ala, 474Gly, 477Val, 480Pro, 481Asp, 482Asn, 483Phe, 484Tyr, 486Leu, 487Val, 488Ala, and 492Glu.
39. The AibRS according to any preceding embodiment; wherein the variant comprises at least 434 amino acids selected from a list consisting of: 1 Met, 3Met, 4Asp, 5Met, 7Thr, 8Arg, 9Met, 10Phe, 11Lys, 12Glu, 13Glu, 14Gly, 15Trp, 16Ile, 17Arg, 18Lys, 20Cys, 21 Lys, 23Cys, 24Gly, 25Lys, 27Phe, 28Trp, 29Thr, 30Leu, 31Asp, 32Pro, 33Asp, 34Arg, 35Glu, 36Thr, 37Cys, 38Gly, 39Asp, 40Pro, 41 Pro, 42Cys, 43Asp, 44Glu, 45Tyr, 47Phe, 48Ile, 49Gly, 50Lys, 51 Pro, 52Gly, 53Ile, 54Pro, 55Lys, 56Lys, 57Tyr, 58Thr, 59Leu, 61Glu, 62Met, 63Arg, 64Glu, 65Lys, 66Phe, 67Leu, 68Ser, 69Phe, 70Phe, 71Glu, 74Gly, 75His, 76Gly, 77Arg, 78Val, 79Lys, 80Arg, 81Tyr, 82Pro, 83Val, 84Leu, 85Pro, 86Arg, 87Trp, 88Arg, 89Asp, 90Asp, 91Val, 92Leu, 93Leu, 94Val, 95Gly, 96Ala, 97Ser, 98Ile, 99Met, 100Asp, 101Phe, 102Gln, 103Pro, 104Trp, 105Val, 106Ile, 107Ser, 108Gly, 109Glu, 110Ala, 111Asp, 112Pro, 113Pro, 114Ala, 115Asn, 116Pro, 117Leu, 118Thr, 119Ile, 120Ser, 121Gln, 122Pro, 123Ser, 124Ile, 125Arg, 126Phe, 127Thr, 128Asp, 129Ile, 130Asp, 131Asn, 132Val, 133GIy, 134Ile, 135Thr, 136GIy, 137Arg, 138His, 139Phe, 140Thr, 141Ile, 142Phe, 143Glu, 144Met, 145Met, 146Ala, 147His, 148His, 149Ala, 150Phe, 151Asn, 152Tyr, 153Pro, 154GIy, 155Lys, 156Pro, 157Ile, 158Tyr, 159Trp, 160Met, 161Asp, 162Glu, 163Thr, 164Val, 165Glu, 166Leu, 167Ala, 168Phe, 169Glu, 170Phe, 171Phe, 172Thr, 173Lys, 175Leu, 176GIy, 177Met, 178Lys, 179Pro, 180Glu, 181Asp, 182Ile, 183Thr, 184Phe, 185Lys, 186Glu, 187Asn, 188Pro, 189Trp, 190Ala, 191Gly, 192GIy, 193GIy, 194Asn, 195Ala, 196GIy, 197Pro, 198Ala, 199Phe, 200Glu, 201Val, 202Leu, 203Tyr, 204Arg, 205GIy, 206Leu, 207Glu, 208Val, 209Ala, 210Thr, 211Leu, 212Val, 213Phe, 214Met, 215GIn, 216Tyr, 217Lys, 219Ala, 220Pro, 223Ala, 227GIn, 228Val, 229Val, 231Ile, 232Lys, 233Gly, 234Asp, 236Tyr, 237Val, 238Pro, 239Met, 241Thr, 243Val, 244Val, 245Asp, 246Thr, 247Gly, 248Tyr, 249GIy, 250Leu, 251Glu, 252Arg, 253Leu, 254Val, 255Trp, 256Met, 257Ser, 258GIn, 259GIy, 260Thr, 261 Pro, 262Thr, 263Ala, 264Tyr, 265Asp, 266Ala, 267Val, 268Leu, 269GIy, 270Tyr, 271Val, 272Val, 273Glu, 274Pro, 275Leu, 276Lys, 278Met, 279Ala, 280Gly, 282Glu, 283Lys, 284Ile, 285Asp, 288Ile, 289Leu, 290Met, 291Glu, 292Asn, 293Ser, 294Arg, 295Leu, 296Ala, 297GIy, 298Met, 299Phe, 300Asp, 301Ile, 302Glu, 303Asp, 304Met, 305GIy, 306Asp, 307Leu, 308Arg, 310Leu, 311Arg, 314Val, 315Ala, 317Arg, 318Val, 319GIy, 320Ile, 321 Ser, 322Val, 323Glu, 324Glu, 325Leu, 326Glu, 330Arg, 331 Pro, 332Tyr, 333Glu, 334Leu, 335Ile, 336Tyr, 337Ala, 338Ile, 339Ala, 340Asp, 341 His, 342Thr, 343Lys, 344Ala, 345Leu, 346Thr, 347Phe, 348Met, 349Leu, 350Ala, 351Asp, 352Gly, 353Val, 354Ile, 355Pro, 356Ser, 357Asn, 358Val, 359Lys, 360Ala, 361Gly, 362Tyr, 363Leu, 364Ala, 365Arg, 366Leu, 367Leu, 368Ile, 369Arg, 370Lys, 371Ser, 372Ile, 373Arg, 374His, 375Leu, 376Arg, 377Glu, 378Leu, 379GIy, 380Leu, 381Glu, 383Pro, 384Leu, 385Ser, 386Glu, 387Ile, 388Val, 389Ala, 390Met, 391 His, 392Ile, 393Lys, 394Glu, 395Leu, 398Thr, 399Phe, 400Pro, 401Glu, 402Phe, 403Lys, 404Glu, 405Met, 406Glu, 407Asp, 408Val, 409Ile, 410Leu, 411Asp, 412Ile, 415Val, 416Glu, 417Glu, 418Lys, 419Arg, 420Tyr, 422Glu, 423Thr, 424Leu, 426Arg, 427Gly, 428Ser, 430Leu, 431Val, 433Arg, 434Glu, 435Ile, 437Lys, 438Leu, 439Lys, 440Lys, 442Gly, 445Glu, 447Pro, 448Leu, 449Glu, 450Lys, 451 Leu, 452Ile, 453Leu, 454Phe, 455Tyr, 456Glu, 457Ser, 458His, 459Gly, 460Leu, 461Thr, 462Pro, 463Glu, 464Ile, 465Val, 467Glu, 468Ile, 469Ala, 470Glu, 471 Lys, 472Glu, 473Gly, 475Lys, 476Val, 478Ile, 479Pro, 480Asp, 481Asn, 482Phe, 483Tyr, 484Ser, 485Leu, 486Val, 487Ala, 488Lys, 490Ala, and 491Glu.
40. The AibRS according to any preceding embodiment; wherein the variant is further characterised by:
   position 192 is Trp, His, Val, lie, or Leu
   position 193 is Ala, Leu, lie, or Gly
   position 213 is Thr, Ser, Cys, or Ala
   position 216 is Phe or Trp
   position 217 is Met, lie, or Leu
   position 249 is Thr, Ser, Val, or Phe
   position 360 is Asn or Ala,
   position 459 is Glu or Ala
41. The AibRS according to any preceding embodiment; wherein the variant is further characterised by:
   position 192 is His, Val, lie, or Leu
   position 193 is Ala, Leu, lie, or Gly
   position 213 is Thr, Ser, Cys, or Ala
   position 216 is Phe or Trp
   position 217 is Met, lie, or Leu
   position 249 is Thr, Ser, Val, or Phe
   position 360 is Ala,
   position 459 is Ala
42. The AibRS according to any preceding embodiment; wherein the variant further comprises one or more amino acids selected from a list consisting of: 192Trp, 192His, 192Val, 192Ile, 192Leu, 193Ala, 193Leu, 193Ile, 193Gly, 213Thr, 213Ser, 213Cys, 213Ala, 216Phe, 216Trp, 217Met, 217Ile, 217Leu, 249Thr, 249Ser, 249Val, 249Phe, 360Asn, 360Ala, 459Glu, and 459Ala.
43. The AibRS according to any preceding embodiment; wherein the variant further comprises two or more amino acids selected from a list consisting of: 192Trp, 192His, 192Val, 192Ile, 192Leu, 193Ala, 193Leu, 193Ile, 193Gly, 213Thr, 213Ser, 213Cys, 213Ala, 216Phe, 216Trp, 217Met, 217Ile, 217Leu, 249Thr, 249Ser, 249Val, 249Phe, 360Asn, 360Ala, 459Glu, and 459Ala.
44. The AibRS according to any preceding embodiment; wherein the variant further comprises three or more amino acids selected from a list consisting of: 192Trp, 192His, 192Val, 192Ile, 192Leu, 193Ala, 193Leu, 193Ile, 193Gly, 213Thr, 213Ser, 213Cys, 213Ala, 216Phe, 216Trp, 217Met, 217Ile, 217Leu, 249Thr, 249Ser, 249Val, 249Phe, 360Asn, 360Ala, 459Glu, and 459Ala.
45. The AibRS according to any preceding embodiment; wherein the variant further comprises one or more amino acids selected from a list consisting of: 192His, 192Val, 192Ile, 192Leu, 193Leu, 193Ile, 193Gly, 213Ser, 213Cys, 213Ala, 216Trp, 217Ile, 217Leu, 249Ser, 249Val, 249Phe, 360Ala, and 459Ala.
46. The AibRS according to any preceding embodiment; wherein the variant further comprises two or more amino acids selected from a list consisting of: 192His, 192Val, 192Ile, 192Leu, 193Leu, 193Ile, 193Gly, 213Ser, 213Cys, 213Ala, 216Trp, 217Ile, 217Leu, 249Ser, 249Val, 249Phe, 360Ala, and 459Ala.
47. The AibRS according to any preceding embodiment; wherein the variant further comprises three or more amino acids selected from a list consisting of: 192His, 192Val, 192Ile, 192Leu, 193Leu, 193Ile, 193Gly, 213Ser, 213Cys, 213Ala, 216Trp, 217Ile, 217Leu, 249Ser, 249Val, 249Phe, 360Ala, and 459Ala.
48. The AibRS according to any preceding embodiment; wherein the variant is selected from a list consisting of: SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.
49. The AibRS according to any preceding embodiment; wherein the variant is selected from a list consisting of: SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.
50. The AibRS according to any preceding embodiment; wherein the AibRS is derived from an archaeal bacterium.
51. The AibRS according to any preceding embodiment; wherein the AibRS is derived from Pyrococcus horikoshii.
52. The AibRS according to any preceding embodiment; wherein the AibRS is configured to perform the function of aminoacylating a tRNA with Aib.
53. The AibRS according to any preceding embodiment; wherein the tRNA is a suppressor tRNA.
54. The AibRS according to any preceding embodiment; wherein the tRNA comprises one or more anticodons that encodes Aib.
55. The AibRS according to any preceding embodiment; wherein the anticodon of the tRNA that encodes Aib is complementary to a stop codon.
56. The AibRS according to any preceding embodiment; wherein the anticodon that encodes Aib is CTA.
57. The AibRS according to any preceding embodiment; wherein the tRNA is encoded by SEQ ID NO: 3 or a variant hereof.
58. The AibRS according to any preceding embodiment; wherein the variant encoding the tRNA contains a G3A mutation.
59. The AibRS according to any preceding embodiment; wherein the variant encoding the tRNA is at least 50%, at least 60%, at least 70% at least 75%, at least 80%, at least 85%, at least 90%, at least 95%, at least 99%, or at least 100% identical to SEQ ID NO: 3.
60. The AibRS according to any preceding embodiment; wherein the variant encoding the tRNA is SEQ ID NO: 4.
61. The AibRS according to any preceding embodiment; wherein the tRNA is encoded by SEQ ID NO: 4.
62. The AibRS according to any preceding embodiment; wherein the tRNA is derived from an archaeal bacterium.
63. The AibRS according to any preceding embodiment; wherein the tRNA is derived from Pyrococcus horikoshii.
64. The AibRS according to any preceding embodiment; wherein the AibRS is configured to perform the function of aminoacylating a tRNA with Aib in a host cell.
65. The AibRS according to any preceding embodiment; wherein the AibRS is configured to perform the function of aminoacylating a tRNA with Aib in E. coli.
66. The AibRS according to any preceding embodiment; wherein the efficiency of the function is determined by analysing the expression product, i.e. the resulting polypeptide upon translation of the tRNA.
67. The AibRS according to any preceding embodiment; wherein the efficiency is expressed as the incorporation ratio between the resulting polypeptide containing Aib and the amount of resulting polypeptide containing Ala or Aib in the position intended for Aib. 68. The AibRS according to any preceding embodiment; wherein the incorporation ratio is determined using LC-MS and calculated based on the mass spectrum as follows: Incorporation ratio=[Peak intensity]_{Aib-containing polypeptide}/([Peak intensity]_{Aib-containing polypeptide}+[Peak intensity]_{Ala-containing polypeptide})*100%.
69. The AibRS according to any preceding embodiment; wherein the incorporation ratio is at least 1%, at least 5%, at least 10%, at least 15%, at least 20%, at least 25%, at least 30%, at least 35%, at least 40%, at least 45%, at least 50%, at least 55, at least 60%, at least 65%, or at least 70%.
70. The use of an AibRS, according to any preceding embodiment, for the preparation of a resulting polypeptide containing one or more Aib residues.
71. The use of an AibRS, according to any preceding embodiment, wherein the resulting polypeptide containing one or more Aib residues is a GLP-1 analogue.
72. The use of an AibRS according to any preceding embodiment, wherein the resulting polypeptide containing one or more Aib residues comprises SEQ ID NO: 32.
73. A method of producing a compound comprising the steps of:
   a. preparation of a resulting polypeptide containing one or more Aib residues using the AibRS of any preceding embodiment
   b. derivatisation of the resulting polypeptide.
74. A method according to any of the preceding embodiments wherein the resulting polypeptide comprises SEQ ID NO: 32.
75. A method according to any of the preceding embodiments wherein the compound is

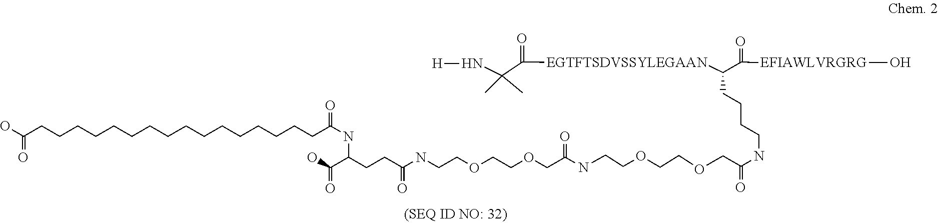

76. A method according to any of the preceding embodiments; wherein the resulting polypeptide comprises SEQ ID NO: 32; and wherein the compound is Chem. 2:

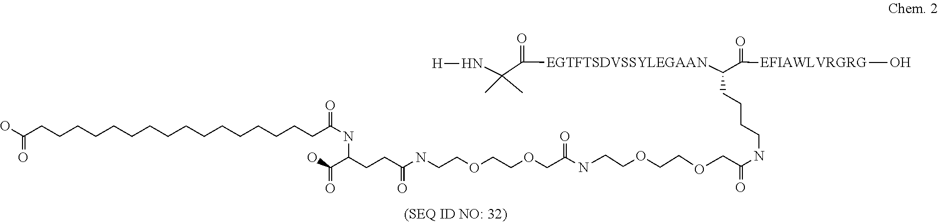

77. A method according to any of the preceding embodiments; wherein the derivatisation introduces a substituent of Chem. 3:

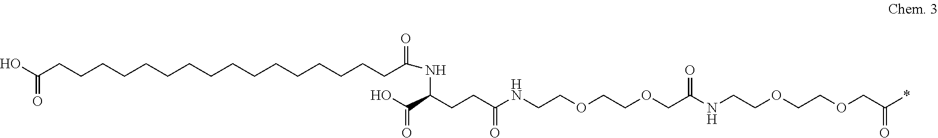 [SMILES: *C(=O)COCCOCCNC(=O)COCCOCCNC(=O)CC[C@H](NC(=O)CCCCCCCCCCCCCCCCC(=O)O)C(=O)O]

### EXAMPLES

### General Procedures

### SDS-PAGE

SDS-PAGE was carried out as standard in the art using 4-12% NuPAGE™ Bis-Tris to analyse expression samples. For preparation of gels and samples the protocols of the supplier (Thermofisher) were applied.

Plasmid Construction for Expression of tRNA^{Aib}, AibRS and Target Polypeptide (i.e. Resulting Polypeptide)

Expression of the tRNA^{Aib}, the AibRS and the target polypeptide (i.e. the resulting polypeptide) was performed from two separate plasmids or a single plasmid. In case of using two separate plasmids, each contained separate selection markers, i.e. amp and kan resistance markers. In case of using a single plasmid, a single marker, i.e. amp, was employed. Expression of the tRNA^{Aib}, the AibRS and the target polypeptide (i.e. the resulting polypeptide) were controlled by separate promoter and terminator sequences, i.e. T7 promotor and terminator to control expression of the target polypeptide, lac promotor and terminator to control expression of the AibRS, and Ipp promotor and terminator to control expression of the tRNA^{Aib}. Preferably the tRNA^{Aib} was expressed for multiple copies of the tDNA, i.e. as two separate 3× concatemers where the expression from each was controlled by an Ipp promoter and terminator as well as a tet promoter and terminator, respectively. Each individual tRNA^{Aib} in the concatemer was spaced by VaIU-VaIX operon linker and IleT and AlaT operon linker to enable processing by RNAses to release the separate tRNA molecules.

E. coli Transformation

Transformation of E. coliwas performed by standard methods according to Sambrook et al. (1989) [Sambrook J, Fritsch E F, Maniatis T.; Molecular Cloning: A Laboratory Manual, 2nd edn; Cold Spring Harbor Laboratory Press: New York; 1989] or by electroporation with a Bio-Rad Gene Pulser set at 25 μF, 200 ohm, and 2.5 kV in 2-mm cuvettes according to Dower et al. (1988) [Dower, W. J., Miller, J. F., & Ragsdale, C. W. (1988) Nucleic Acids Res. 16, 6127-6145]. Transformed cells were selected on LB media supplemented with the appropriate selective antibiotics, i.e. ampicillin and/or canavanine.

### E. Colicultivation

E. coliTKO cells (WO/2010/052335) transformed with the plasmids to be tested were taken either from a frozen stocks or directly from fresh transformations on LB plates (with appropriate antibiotics). Cells were then inoculated into 50 ml tubespin bioreactor P50 filled with 8 ml LB medium plus antibiotics. The cells were grown at 37° C. with shaking at 220 rpm for 5 hours. Cells from the pre-culture were diluted into 20 ml of minimal defined medium (M9) with appropriate antibiotics filled into 125 mL Corning@Disposable Erlenmeyer Flask. Aib were added to a concentration of 10 mM. The cells were cultivated at 37° C. to OD600 to 1.0. Target protein expression was induced by addition of 1 mM IPTG and further cultivated at 30° C. or 37° C. for a minimum of 4 hours up to overnight. Inclusion body fractions were isolated and collected with sonication and centrifugation as described in next paragraph.

### Sample Preparation and LC-MS Methods

A suspension of E. colicells, corresponding to 10 OD, was centrifuged at 13000×g for 5 minutes. The resulting pellet was re-suspended in 1 ml 10 mM Kaliumphoshate pH 5.0 buffer and sonificated for 2 min at 30% amplitude, 5/5″ on/off. After repeated centrifugation at 13000×g for 5 minutes, the pellet fraction was dissolved with 8M urea/100 mM DTT/50 mM CAPS pH 12 buffer and shaken 1 h at RT, 2000×rpm. Samples were filtered with 0.2 μm UPLC filters and analysed by LC-MS using the following setup:

| Parameter | Information |
|---|---|
| MS System | Waters UPLC Xevo G2 Q-TOF (in 3.3.C.19.1) |
| Resolution | 20,000 |
| Ion source | Dual ESI (positive mode), 120° C. |
| Capillary | 3 KV |
| Sample Cone | 35 V |
| Desolvation | 380° C. |
| Temperature | |
| Cone Gas (L/h) | 38 |
| Desolvation Gas | 800 |
| (L/h) | |
| Range m/z | m/z: 500 to 4,000 (200-4,000) |
| Column | Acquity UPLC BEH300 C4 1.7 um |
| | (Size: 2.1 × 50 mm) |
| | (Waters Part# 186004495) |
| Column | 80° C. (45° C.) |
| temperature | |
| Flow Rate | 0.2-0.4 mL/min |
| Pressure limit | 15000 psi |
| Buffer A | Formic acid (0.1% v/v) in water (Fisher LC118-1) |
| Buffer B | Formic acid(0.1% v/v) in acetonitrile (Fisher LC120-1) |
| Buffer C | Trifluoroacetic acid (0.05% v/v) in water |
| Buffer D | Trifluoroacetic acid(0.04% v/v) in acetonitrile |
| Gradient/RunTime | General gradient: |
| | 5% B for 2.5 min, 5-95% B in 5 min, keep 95% B for |
| | 0.5 min, reduce to 5% B in 0.1 min and keep for |
| | 1.9 min, Total runtime: 10 min |

tRNA Synthetase and tRNA Suitable for 2-Aminoisobutyric Acid Incorporation in a Polypeptide During Translation

Methodologies that allow the systematic addition of unnatural amino acids to the genetic code of E. coli, yeast and mammalian cells have previously been reported [Wals K. and Ovaa H; Unnatural amino acid incorporation in E. coli: current and future applications in the design of therapeutic proteins; Front Chem. Apr 1; 2:15 (2014)], [Wang 0, and Wang L; Genetic incorporation of unnatural amino acids into proteins in yeast; Methods Mol. Biol;794: 199-213 (2012)] and [Schmied WH, Elsasser SJ; Efficient multisite unnatural amino acid incorporation in mammalian cells via optimized pyrrolysyl tRNA synthetase/tRNA expression and engineered eRF1; Am Chem Soc. Nov 5;136(44):15577-83 (2014)]. The methods are based on the selection of an orthogonal tRNA/RS pair isolated from a distant organism, that enable the cells to incorporate a given amino acid in response to a unique codon, without or with reduced cross reacting with the endogenous host tRNAs, RS or amino acids.

In order to specifically incorporate 2-Aminoisobutyric acid (Aib) into a polypeptide, an RS, capable of charging tRNA with Aib, can be developed and utilised together with other translation machinery such as that endogenously part of a host cell (e.g. E. coli). The RS capable of charging tRNA with Aib will in the following be referred to as 2-Aminoisobutyric acid-tRNA synthetase (AibRS). The tRNA molecule charged with Aib will in the following be referred to as tRNA^{Aib}. The AibRS and the tRNA^{Aib} will in the following collectively be referred to as an AibRS/tRNA^{Aib} pair.

The AibRS and the tRNA^{Aib} may conveniently be derived from an AlaRS and tRNA^{Ala} respectively, as Ala shares structural similarities with Aib. The AlaRS and tRNA^{Ala} may in theory originate from any organism. In this case, the AibRS and tRNA^{Aib} were derived from a wild-type AlaRS and wild-type tRNA^{Ala} from an archaeal bacterium, Pyrococcus horikoshii (ph) (referred to as phAlaRS(wt) (SEQ ID NO: 1) and phtRNA^{Ala}(wt) (SEQ ID NO: 2), respectively). In this case, tRNA^{Aib} optimisation and AibRS development took place using E. colias a model system, albeit any host cell could be used for this purpose in theory.

Optimisation of the tRNA^{Aib}

### Example 1

The tRNA^{Aib} may in principle be any tRNA (e.g. tRNA^{A}1a) that is cognate to AibRS. However, in order to improve the efficiency of the translation process incorporating an Aib residue, the tRNA can be optimised so that the formation of undesired translation products, e.g. polypeptides containing Ala rather than Aib, is reduced.

In order to avoid the unwanted incorporation of Aib into all positions coding for Ala, the anticodon in phtRNA^{Ala}(wt) (SEQ ID NO: 2) was changed to CTA, reversely complementing with TAG, which is an amber stop codon, thus resulting in a suppressor phtRNA^{Ala} (SEQ ID NO: 3). Any stop codon could in principle be used for this purpose.

The suppressor phtRNA^{Ala} (SEQ ID NO: 3) is not orthogonal to the endogenous E. coli translation system, meaning that suppressor phtRNA^{Ala} (SEQ ID NO: 3) can be recognized by endogenous AlaRS of E. coli (ecAlaRS) and aminoacylated with Ala rather than Aib. In order to favour aminacylation with Aib, mutations can be introduced in the phtRNA^{Ala} (SEQ ID NO: 3) to make it orthogonal to ecAlaRS, while maintaining its complementarity to AibRS. While such mutations may offer improved efficiency, they are in principle not needed for the invention to work. Aligning sequences from the public domain (i.e. Genbank or EMBL), the G3 and T72 DNA nucleotides forming a G-U pair in phtRNA^{Ala}(wt) (SEQ ID NO: 2) secondary structure is conserved in tRNA^{Ala}across different species. In addition it has been demonstrated for other archaebacteria that a mutation of G3A in the tRNA^{Ala} of Aquifex aeolicus makes the tRNA^{Ala} unrecognisable to any AlaRS [MA Swairjo et al.; Alanyl-tRNA Synthetase Crystal Structure, and Design for Acceptor-Stem Recognition; Molecular Cell, Vol. 13, 829-841, (2004)]. Inspired by the observations in Aquifex aeolicus, a suppressor phtRNA^{Ala} (SEQ ID NO: 3) containing a G3A mutation was prepared resulting in a tRNA^{Aib} (SEQ ID NO: 4).

The orthogonality of suppressor phtRNA^{Ala} (SEQ ID NO: 3) and tRNA^{Aib} (SEQ ID NO: 4) was tested. In a first sample, the E. coli cells were modified by standard plasmid transformation were exogenous DNA encoding suppressor phtRNA^{Ala} (SEQ ID NO: 3) and the model polypeptide MS-(Aib)-hsLeptin (SEQ ID NO: 5) were transferred into the cell. In the second sample, the E. coli cells were modified by standard plasmid transformation, were exogenous DNA encoding tRNA^{Aib} (SEQ ID NO: 4) and the model polypeptide MS-(Aib)-hsLeptin (SEQ ID NO: 5) were transferred into the cell. In theory, the full-length model polypeptides would be expressed (albeit the model polypeptide MS-(Aib)-hsLeptin would contain Ala rather than Aib) if the endogenous ecAlaRS recognised the exogenous suppressor phtRNA^{Ala} (SEQ ID NO: 3). The cell were cultivated according to the general procedures and analysed by SDS-PAGE. The analysis showed that full-length model polypeptide was expressed in the cells containing suppressor phtRNA^{Ala} (SEQ ID NO: 3), while no (or very little) full-length model polypeptide was expressed in the cells containing DNA encoding tRNA^{Aib} (SEQ ID NO: 4), and thus the tRNA^{Aib} (SEQ ID NO: 4) was found to be recognised by ecAlaRS with a significantly reduced efficiency.

### Development of 2-Aminoisobutyric Acid-tRNA Synthetase (AibRS)

### Example 2

In comparison to Ala, 2-Aminoisobutyric acid (Aib) (Chem. 1) contains an extra methyl group at the alpha-carbon position, which makes the Aib unable to interact with phAlaRS(wt) due to steric hindrance.

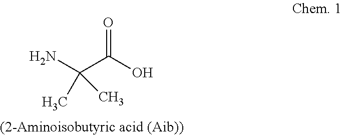

The AlaRS class II core catalytic domain of phAlaRS(wt), which contains the Ala binding pocket, is the amino acid sequence spanning position 1 to 263 of the wild-type sequence and is part of an N-terminal domain comprising the Ala binding and tRNA^{Ala} binding functions, which is referred to as “aa1-495” in the following. The editing and the oligomerization domain (aa496-915) were omitted from all constructs in this study, and thus all RS constructs in the following is based on the aa1-495 portion of the phAlaRS(wt) (SEQ ID NO: 6). A phAlaRS(wt) crystal structure of the N-terminal domains (aa1-752) is published by M. Sokabe et al. (2009) [Sokabe M, Ose T, Nakamura A, Tokunaga K, Nureki O, Yao M, Tanaka I; The structure of alanyl-tRNA synthetase with editing domain; PNAS 106 (27): 11028-11033 (2009)].

The alanine binding pocket of the AlaRS was calculated based on residues having atoms closer than 5 angstroms to the alanine C-alpha carbon atom in the crystal structure 2ZZG (Crystal structure of alanyl-tRNA synthetase in complex with 5″-O-(N-(L-alanyl)-sulfamyoxyl) adenine without oligomerization domain). Distances were calculated in standard structure visualisation software, particularly Accelrys VL Viewer, by loading the 2ZZG.pdb file, selecting the A5A999.CA atom, and then selecting atoms within 5 angstroms. These atoms belonged to A99, M147, W192, T213, V215, D248, T249, and G250. In particular W192 and V215 were in close proximity to the putative location of the extra methyl group of Aib. It was speculated that the extra methyl group of Aib could be accommodated by mutations in these two positions. Mutants of the aa1-495 portion of the phAlaRS(wt) (SEQ ID NO: 6) were prepared, specifically, two mutations in position 192, i.e. W192H and W192F, were tested, and one mutation in position 215, i.e. V215G, was tested alone and in combination with W1 92H, resulting in a total of four mutants. Also, the wild-type aa1-495 portion of the phAlaRS(wt) (SEQ ID NO: 6) was tested.

TABLE 1
Aminoacyl-tRNA synthetase mutants of aa1-495
portion of the phAIaRS(wt) (SEQ ID NO: 6)

| SEQ ID NO | Mutations |
|---|---|
| 7 | V215G |
| 8 | W192F |
| 9 | W192H |
| 10 | W192H; V215G |
| 6 | No mutations (wild type) |

The mutants were transformed into E. coli TKO cells together with one copy of suppressor phtRNA^{Ala} (SEQ ID NO: 3) and a polynucleotide encoding the model polypeptide MS-(Aib)-hsLeptin (SEQ ID NO: 5) by standard plasmid transformation methods. The E. coli cells were cultivated according to the general procedures. The isolated inclusion body fractions were analysed with SDS-PAGE. For all mutants including the V215G and/or W192H mutation (i.e. SEQ ID NO: 7, SEQ ID NO: 9, and SEQ ID NO: 10) a band at ~16 kDa, of varying intensity, was observed (no band was observed for SEQ ID NO: 8 or the wild type). Solubilized inclusion bodies of were analysed using LC-MS according to the general procedures, and it was confirmed that the band in sample with SEQ ID NO: 7 represented a small portion of the expressed polypeptide MS-(Aib)-hsLeptin (SEQ ID NO: 5) (found [m/1]=16198.6; calculated [m/1]=16198.4). The efficiency of the mutant was expressed as the incorporation ratio and calculated as described in Example 3. The incorporation ratio for SEQ ID NO: 7 was =<10%.

### Example 3

For the archaea Archaeoglobus fulgidus it has been reported that the tRNA^{Ala}aminoacylation mechanism may be based on a single G-U pair, corresponding to the G3 and T72 in the DNA polynucleotide, and that key residues of the AlaRS involved in the interaction with this pair are N359 & D450, and that for AlaRS of Pyrococcus horikoshiithe two corresponding key residues are N360 and E459. Furthermore, it has been reported that these key residues in AlaRS are conserved across archaea domain [M. Naganuma et al., Nature 510, pages 507-511 (2014)].

A mutant of aa1-495 of phAlaRS(wt) containing the W1 92H, V215G, N360A and E459A mutations (SEQ ID NO: 11) were transformed, according to general procedures, into E. coli TKO cells on a plasmid containing 6 copies of tRNA^{Aib} (SEQ ID NO: 4) and a polynucleotide encoding a model polypeptide IL21-H(Aib)-GLP-1 (SEQ ID NO: 31) comprising an inclusion body-inducing region from IL-21 protein at positions 1-24, and an Aib-containing GLP-1 analogue region. The Aib-containing GLP-1 region was identical to the polypeptide backbone of semaglutide (SEQ ID NO: 32) [Lau J. et. al, Discovery of the once-weekly glucagon-like peptide-1 (GLP-1) analogue semaglutide, J. Med. Chem., 2015; 58:7370-7380]. The E. coli cells were cultivated according to general procedures and analysed by SDS-PAGE. A strong band at ~8 kDa was identified, and LC-MS confirmed that the model polypeptide IL21-H(Aib)-GLP-1 (SEQ ID NO: 31) had been expressed (found [m/1]=8324.6; calculated [m/1]=8324.3 The efficiency of the Aib function was expressed as a incorporation ratio between the amount of resulting polypeptide containing Aib and the amount of resulting polypeptide containing Aib or Ala in the position encoding for Aib, and calculated based on the mass spectrum as follows: Incorporation ratio=[Peak intensity]_{Aib-containing polypeptide}/([Peak intensity]_{Aib-containing polypeptide}+[Peak intensity]_{Ala-containing polypeptide})*1 00%. The incorporation ratio for SEQ ID NO: 11 was calculated to be 63%.

### Example 4

A mutant of aa1-495 of phAlaRS(wt) containing the W192H, V215G, M2171, N360A and E459A (SEQ ID NO: 12) was prepared and tested according to the procedures described in Example 3. LC-MS confirmed that the model polypeptide IL21-H(Aib)-GLP-1 (SEQ ID NO: 12) had been expressed (found [m/1]=8324,6; calculated [m/1]=8324.3. The calculated incorporation ratio was 82%.

### Example 5

A mutant of aa1-495 of phAlaRS(wt) containing the W192H, V215G, M217L, N360A, and E459A (SEQ ID NO: 13) was prepared and tested according to the procedures described in Example 3. LC-MS confirmed that the model polypeptide IL21-H(Aib)-GLP-1 (SEQ ID NO: 13) had been expressed (found [m/1]=8324.6; calculated [m/1]=8324.3. The calculated incorporation ratio was 66%.

### Example 6

A mutant of aa1-495 of phAlaRS(wt) containing the W192H, V215G, A193L, N360A, and E459A (SEQ ID NO: 14) was prepared and tested according to the procedures described in Example 3. LC-MS confirmed that the model polypeptide IL21-H(Aib)-GLP-1 (SEQ ID NO: 14) had been expressed (found [m/1]=8324,6; calculated [m/1]=8324.3. The calculated incorporation ratio was 71%.

### Example 7

A mutant of aa1-495 of phAlaRS(wt) containing the W192H, V215G, F216W, N360A, and E459A (SEQ ID NO: 15) was prepared and tested according to the procedures described in Example 3. LC-MS confirmed that the model polypeptide IL21-H(Aib)-GLP-1 (SEQ ID NO: 15) had been expressed (found [m/1]=8324.6; calculated [m/1]=8324.3. The calculated incorporation ratio was 66%.

### Example 8

A mutant of aa1-495 of phAlaRS(wt) containing the W192H, V215G, A193L, F216W, N360A, and E459A (SEQ ID NO: 16) was prepared and tested according to the procedures described in Example 3. LC-MS confirmed that the model polypeptide IL21-H(Aib)-GLP-1 (SEQ ID NO: 16) had been expressed (found [m/1]=8324.6; calculated [m/1]=8324.3. The calculated incorporation ratio was 63%.

### Example 9

A mutant of aa1-495 of phAlaRS(wt) containing the W192H, V215G, and A1931, N360A, and E459A (SEQ ID NO: 17) was prepared and tested according to the procedures described in Example 3. LC-MS confirmed that the model polypeptide IL21-H(Aib)-GLP-1 (SEQ ID NO: 16) had been expressed (found [m/1]=8324.8; calculated [m/1]=8324.3. The calculated incorporation ratio was 60%.

### Example 10

A mutant of aa1-495 of phAlaRS(wt) containing the W192H, V215G, A1931, M2171, N360A, and E459A (SEQ ID NO: 18) was prepared and tested according to the procedures described in Example 3. LC-MS confirmed that the model polypeptide IL21-H(Aib)-GLP-1 (SEQ ID NO: 16) had been expressed (found [m/1]=8324.6; calculated [m/1]=8324.3. The calculated incorporation ratio was 72%.

### Example 11

A mutant of aa1-495 of phAlaRS(wt) containing the W192H, V215G, A193L, M2171, N360A, and E459A (SEQ ID NO: 19) was prepared and tested according to the procedures described in Example 3. LC-MS confirmed that the model polypeptide IL21-H(Aib)-GLP-1 (SEQ ID NO: 16) had been expressed (found [m/1]=8324.6; calculated [m/1]=8324.3. The calculated incorporation ratio was 82%.

### Example 12

A mutant of aa1-495 of phAlaRS(wt) containing W192H, V215G, A1931, M217L, N360A. and E459A (SEQ ID NO: 20) was prepared and tested according to the procedures described in Example 3. LC-MS confirmed that the model polypeptide IL21-H(Aib)-GLP-1 (SEQ ID NO: 16) had been expressed (found [m/1]=8324.8; calculated [m/1]=8324.3. The calculated incorporation ratio was 51%.

### Example 13

A mutant of aa1-495 of phAlaRS(wt) containing W192H, V215G, A193L, M217L, N360A, and E459A (SEQ ID NO: 21) was prepared and tested according to the procedures described in Example 3. LC-MS confirmed that the model polypeptide IL21-H(Aib)-GLP-1 (SEQ ID NO: 21) had been expressed (found [m/1]=8324.8; calculated [m/1]=8324.3. The calculated incorporation ratio was 57%.

### Example 14

A mutant of aa1-495 of phAlaRS(wt) containing W192V, V215G, M2171, N360A, and E459A (SEQ ID NO: 22) was prepared and tested according to the procedures described in Example 3. LC-MS confirmed that the model polypeptide IL21-H(Aib)-GLP-1 (SEQ ID NO: 16) had been expressed (found [m/1]=8324.4; calculated [m/1]=8324.3. The calculated incorporation ratio was 77%.

### Example 15

A mutant of aa1-495 of phAlaRS(wt) containing W1921, V215G, M2171, N360A, and E459A (SEQ ID NO: 23) was prepared and tested according to the procedures described in Example 3. LC-MS confirmed that the model polypeptide IL21-H(Aib)-GLP-1 (SEQ ID NO: 16) had been expressed (found [m/1]=8324.4; calculated [m/1]=8324.3. The calculated incorporation ratio was 74%.

### Example 16

A mutant of aa1-495 of phAlaRS(wt) containing W192L, V215G, M2171, N360A, and E459A (SEQ ID NO: 24) was prepared and tested according to the procedures described in Example 3. LC-MS confirmed that the model polypeptide IL21-H(Aib)-GLP-1 (SEQ ID NO: 16) had been expressed (found [m/1]=8325.0; calculated [m/1]=8324.3. The calculated incorporation ratio was 54%.

### Example 17

A mutant of aa1-495 of phAlaRS(wt) containing W192H, A193G, V215G, M2171, N360A, and E459A (SEQ ID NO: 25) was prepared and tested according to the procedures described in Example 3. LC-MS confirmed that the model polypeptide IL21-H(Aib)-GLP-1 (SEQ ID NO: 16) had been expressed (found [m/1]=8324.4; calculated [m/1]=8324.3. The calculated incorporation ratio was 86%.

### Example 18

A mutant of aa1-495 of phAlaRS(wt) containing W192H, T213S, V215G, N360A, and E459A (SEQ ID NO: 26) was prepared and tested according to the procedures described in Example 3. LC-MS confirmed that the model polypeptide IL21-H(Aib)-GLP-1 (SEQ ID NO: 16) had been expressed (found [m/1]=8324.2; calculated [m/1]=8324.3. The calculated incorporation ratio was 56%.

### Example 19

A mutant of aa1-495 of phAlaRS(wt) containing W192H, V215G, T249S, N360A, and E459A (SEQ ID NO: 27) was prepared and tested according to the procedures described in Example 3. LC-MS confirmed that the model polypeptide IL21-H(Aib)-GLP-1 (SEQ ID NO: 16) had been expressed (found [m/1]=8325.0; calculated [m/1]=8324.3. The calculated incorporation ratio was 67%.

### Example 20

A mutant of aa1-495 of phAlaRS(wt) containing W192H, V215G, T249V, N360A, and E459A (SEQ ID NO: 28) was prepared and tested according to the procedures described in Example 3. LC-MS confirmed that the model polypeptide IL21-H(Aib)-GLP-1 (SEQ ID NO: 16) had been expressed (found [m/1]=8324.8; calculated [m/1]=8324.3. The calculated incorporation ratio was 78%.

### Example 21

A mutant of aa1-495 of phAlaRS(wt) containing W192H, T213C, V215G, T249V, N360A, and E459A (SEQ ID NO: 29) was prepared and tested according to the procedures described in Example 3. LC-MS confirmed that the model polypeptide IL21-H(Aib)-GLP-1 (SEQ ID NO: 16) had been expressed (found [m/1]=8324.8; calculated [m/1]=8324.3. The calculated incorporation ratio was 82%.

### Example 22

A mutant of aa1-495 of phAlaRS(wt) containing W192H, T213A, V215G, T249F, N360A, and E459A (SEQ ID NO: 30) was prepared and tested according to the procedures described in Example 3. LC-MS confirmed that the model polypeptide IL21-H(Aib)-GLP-1 (SEQ ID NO: 16) had been expressed (found [m/1]=8324.6; calculated [m/1]=8324.3. The calculated incorporation ratio was 83%.

### Example 23

A consensus sequence for the archaean AlaRS was made by extracting the 100 sequences most identical to the aa1-495 portion of the phAlaRS(wt) (SEQ ID NO: 6). From the NCBI sequence database using SEQ ID NO: 6 as query against the “All non-redundant GenBank CDS translations+PDB+SwissProt+PIR+PRF” and using the NCBI online blastp suite. The extracted sequences were trimmed N-terminally and C-terminally to the same length as SEQ ID NO: 6 (to remove N-terminal extensions and the C-terminal editing domain) and then aligned using standard alignment algorithms, particularly the Geneious® 10.2.2 software using Multiple Align with Blosum62 matrix. Three consensus sequences were created by setting a threshold of identity as 85%, 75% or 50%, respectively, across all sequences in the alignment to indicate whether the amino acid position was conserved or not. Then a given amino acid position in the consensus sequence was denoted as either a specific amino acid—if conserved above the set threshold—or otherwise Xaa for any amino acid—if not conserved above the threshold. The consensus sequences are provided Table 2. The consensus sequence may change over time as the NCBI genome database is updated regularly.

TABLE 2
Consensus sequences of archaean AIaRS based on SEQ ID NO: 6

| | | Relative |
|---|---|---|
| Threshold | | amount of |
| of | | conserved |
| identity | Sequence¹ | positions |
| 85% | Formula I: | 41% |
| | Xaa-Xaa-Xaa-Xaa-Xaa-Xaa-Xaa-Xaa- | |
| | Xaa-Phe-Xaa-Glu-Xaa-Gly-Xaa-Xaa-Xaa- | |
| | Lys-Xaa-Cys-Xaa-Xaa-Cys-Gly-Xaa-Xaa- | |
| | Phe-Trp-Thr-Xaa-Xaa-Xaa-Xaa-Arg-Xaa- | |
| | Xaa-Cys-Gly-Asp-Xaa-Pro-Cys-Xaa-Xaa-Tyr- | |
| | Xaa-Phe-Ile-Gly-Xaa-Pro-Xaa-Xaa-Xaa- | |
| | Xaa-Xaa-Xaa-Xaa-Leu-Xaa-Glu-Xaa-Arg- | |
| | Xaa-Xaa-Phe-Xaa-Xaa-Phe-Phe-Glu-Xaa- | |
| | Xaa-Xaa-His-Xaa-Xaa-Xaa-Xaa-Arg-Tyr- | |
| | Pro-Val-Xaa-Xaa-Arg-Trp-Arg-Asp-Asp-Val- | |
| | Xaa-Leu-Val-Gly-Ala-Ser-Ile-Xaa-Asp-Phe- | |
| | Gln-Pro-Trp-Val-Xaa-Xaa-Gly-Xaa-Xaa- | |
| | Xaa-Pro-Pro-Ala-Asn-Pro-Leu-Xaa-Ile- | |
| | Ser-Gln-Pro-Xaa-Ile-Arg-Xaa-Xaa-Asp- | |
| | Xaa-Asp-Xaa-Val-Gly-Xaa-Xaa-Gly- | |
| | Arg-His-Xaa-Thr-Xaa-Phe-Glu-Met-Met- | |
| | Ala-His-His-Ala-Phe-Asn-Xaa-Xaa-Xaa-Xaa- | |
| | Xaa-Xaa-Tyr-Trp-Xaa-Xaa-Glu-Thr-Val- | |
| | Xaa-Xaa-Xaa-Xaa-Xaa-Phe-Xaa-Xaa-Xaa- | |
| | Xaa-Xaa-Xaa-Xaa-Xaa-Xaa-Xaa-Xaa-Ile- | |
| | Thr-Phe-Xaa-Glu-Xaa-Xaa-Trp-Xaa-Gly- | |
| | Gly-Gly-Asn-Ala-Gly-Xaa-Xaa-Xaa-Glu-Val- | |
| | Xaa-Xaa-Xaa-Gly-Xaa-Glu-Xaa-Ala-Thr- | |
| | Leu-Val-Phe-Met-Xaa-Tyr-Xaa-Xaa-Xaa-Xaa- | |
| | Xaa-Xaa-Xaa-Xaa-Xaa-Xaa-Xaa-Xaa-Xaa- | |
| | Xaa-Xaa-Xaa-Xaa-Xaa-Xaa-Xaa-Xaa-Pro- | |
| | Xaa-Xaa-Xaa-Xaa-Xaa-Val-Asp-Thr-Gly- | |
| | Tyr-Gly-Leu-Glu-Arg-Xaa-Xaa-Trp-Xaa- | |
| | Ser-Xaa-Gly-Xaa-Pro-Thr-Xaa-Tyr-Asp- | |
| | Ala-Xaa-Xaa-Xaa-Xaa-Xaa-Xaa-Xaa-Xaa- | |
| | Leu-Xaa-Xaa-Xaa-Ala-Gly-Xaa-Xaa-Xaa- | |
| | Xaa-Xaa-Xaa-Xaa-Xaa-Ile-Leu-Xaa-Xaa-Xaa- | |
| | Xaa-Xaa-Xaa-Ala-Gly-Xaa-Xaa-Asp-Xaa- | |
| | Xaa-Xaa-Xaa-Xaa-Xaa-Leu-Xaa-Xaa-Leu- | |
| | Arg-Xaa-Xaa-Val-Ala-Xaa-Xaa-Xaa-Xaa- | |
| | Xaa-Xaa-Xaa-Xaa-Glu-Leu-Xaa-Xaa-Xaa- | |
| | Xaa-Xaa-Pro-Xaa-Glu-Xaa-Xaa-Tyr-Ala-Ile- | |
| | Ala-Asp-His-Thr-Xaa-Xaa-Leu-Xaa-Phe- | |
| | Met-Leu-Xaa-Asp-Gly-Val-Xaa-Pro-Ser-Asn- | |
| | Xaa-Xaa-Ala-Gly-Tyr-Leu-Ala-Arg-Leu-Xaa- | |
| | Ile-Arg-Xaa-Xaa-Xaa-Arg-Xaa-Xaa-Xaa- | |
| | Xaa-Leu-Gly-Xaa-Xaa-Xaa-Pro-Leu-Xaa- | |
| | Xaa-Ile-Xaa-Xaa-Xaa-Xaa-Xaa-Xaa-Xaa- | |
| | Xaa-Xaa-Xaa-Xaa-Xaa-Pro-Glu-Xaa-Xaa- | |
| | Glu-Xaa-Xaa-Xaa-Xaa-Xaa-Xaa-Xaa-Xaa- | |
| | Xaa-Xaa-Xaa-Glu-Glu-Xaa-Xaa-Xaa-Xaa- | |
| | Xaa-Thr-Xaa-Xaa-Arg-Gly-Xaa-Xaa-Xaa- | |
| | Xaa-Xaa-Xaa-Xaa-Xaa-Xaa-Xaa-Xaa-Lys- | |
| | Xaa-Xaa-Gly-Xaa-Xaa-Xaa-Xaa-Pro-Xaa- | |
| | Xaa-Xaa-Leu-Xaa-Xaa-Xaa-Tyr-Xaa-Ser- | |
| | His-Gly-Xaa-Xaa-Pro-Glu-Xaa-Xaa-Xaa- | |
| | Glu-Xaa-Ala-Xaa-Xaa-Xaa-Xaa-Xaa-Xaa- | |
| | Val-Xaa-Xaa-Pro-Asp-Asn-Phe-Tyr-Xaa- | |
| | Xaa-Val-Ala-Xaa-Xaa-Xaa-Xaa-Xaa-Xaa | |
| | (SEQ ID NO: 33) | |
| 75% | Formula II: | 55% |
| | Xaa-Xaa-Xaa-Xaa-Xaa-Xaa-Xaa-Xaa-Xaa- | |
| | Phe-Xaa-Glu-Xaa-Gly-Xaa-Xaa-Arg-Lys- | |
| | Xaa-Cys-Xaa-Xaa-Cys-Gly-Xaa-Xaa-Phe- | |
| | Trp-Thr-Xaa-Asp-Pro-Asp-Arg-Glu-Thr- | |
| | Cys-Gly-Asp-Xaa-Pro-Cys-Asp-Xaa-Tyr-Xaa- | |
| | Phe-Ile-Gly-Xaa-Pro-Xaa-Xaa-Xaa-Xaa- | |
| | Xaa-Tyr-Xaa-Leu-Xaa-Glu-Xaa-Arg-Glu- | |
| | Xaa-Phe-Leu-Xaa-Phe-Phe-Glu-Xaa-Xaa- | |
| | Xaa-His-Xaa-Arg-Xaa-Xaa-Arg-Tyr-Pro-Val- | |
| | Xaa-Xaa-Arg-Trp-Arg-Asp-Asp-Val-Xaa-Leu- | |
| | Val-Gly-Ala-Ser-Ile-Xaa-Asp-Phe-Gln-Pro-Trp- | |
| | Val-Xaa-Ser-Gly-Xaa-Xaa-Xaa-Pro-Pro-Ala- | |
| | Asn-Pro-Leu-Xaa-Ile-Ser-Gln-Pro-Ser-Ile-Arg- | |
| | Xaa-Thr-Asp-Xaa-Asp-Asn-Val-Gly-Xaa- | |
| | Thr-Gly-Arg-His-Xaa-Thr-Xaa-Phe-Glu-Met- | |
| | Met-Ala-His-His-Ala-Phe-Asn-Xaa-Pro-Xaa- | |
| | Xaa-Xaa-Xaa-Tyr-Trp-Xaa-Asp-Glu-Thr-Val- | |
| | Glu-Xaa-Xaa-Xaa-Xaa-Phe-Xaa-Thr-Xaa- | |
| | Xaa-Leu-Xaa-Xaa-Xaa-Xaa-Glu-Xaa-Ile- | |
| | Thr-Phe-Lys-Glu-Xaa-Xaa-Trp- | |
| | Xaa-Gly-Gly-Gly-Asn-Ala-Gly-Pro-Xaa- | |
| | Xaa-Glu-Val-Leu-Xaa-Arg-Gly-Leu- | |
| | Glu-Val-Ala-Thr-Leu-Val-Phe- | |
| | Met-Gln-Tyr-Lys-Xaa-Xaa-Xaa-Xaa- | |
| | Xaa-Xaa-Xaa-Xaa-Xaa-Xaa-Xaa- | |
| | Xaa-Xaa-Xaa-Xaa-Gly-Xaa-Xaa- | |
| | Xaa-Xaa-Pro-Met-Xaa-Xaa-Xaa-Xaa- | |
| | Val-Asp-Thr-Gly-Tyr-Gly-Leu-Glu- | |
| | Arg-Xaa-Val-Trp-Xaa-Ser-Xaa-Gly- | |
| | Thr-Pro-Thr-Ala-Tyr-Asp-Ala-Val-Xaa-Xaa- | |
| | Xaa-Val-Xaa-Xaa-Xaa-Leu-Xaa-Xaa-Xaa- | |
| | Ala-Gly-Xaa-Xaa-Xaa-Xaa-Xaa-Xaa-Xaa- | |
| | Xaa-Ile-Leu-Xaa-Glu-Asn-Ser-Xaa- | |
| | Leu-Ala-Gly-Xaa-Xaa-Asp-Ile-Glu-Xaa-Xaa- | |
| | Xaa-Asp-Leu-Xaa-Xaa-Leu-Arg-Xaa- | |
| | Xaa-Val-Ala-Xaa-Xaa-Xaa-Gly-Ile-Xaa- | |
| | Xaa-Xaa-Glu-Leu-Xaa-Xaa-Xaa-Xaa- | |
| | Xaa-Pro-Xaa-Glu-Xaa-Ile-Tyr-Ala-Ile-Ala- | |
| | Asp-His-Thr-Xaa-Xaa-Leu-Xaa-Phe-Met-Leu- | |
| | Xaa-Asp-Gly-Val-Xaa-Pro-Ser- | |
| | Asn-Val-Lys-Ala-Gly-Tyr-Leu-Ala- | |
| | Arg-Leu-Leu-Ile-Arg-Xaa-Xaa-Xaa-Arg- | |
| | Xaa-Xaa-Xaa-Xaa-Leu-Gly- | |
| | Leu-Xaa-Xaa-Pro-Leu-Xaa-Glu-Ile- | |
| | Xaa-Xaa-Xaa-Xaa-Xaa-Xaa-Xaa-Leu- | |
| | Xaa-Xaa-Xaa-Xaa-Pro-Glu-Xaa-Xaa-Glu- | |
| | Xaa-Xaa-Xaa-Xaa-Ile-Xaa-Xaa-Xaa- | |
| | Xaa-Xaa-Xaa-Glu-Glu-Xaa-Xaa-Tyr-Xaa-Xaa- | |
| | Thr-Leu-Xaa-Arg-Gly-Xaa-Xaa-Leu-Val-Xaa- | |
| | Xaa-Xaa-Xaa-Xaa-Xaa-Leu-Lys-Lys- | |
| | Xaa-Gly-Xaa-Xaa-Glu-Xaa-Pro-Leu- | |
| | Glu-Xaa-Leu-Xaa-Xaa-Xaa- | |
| | Tyr-Xaa-Ser-His-Gly-Xaa-Xaa-Pro-Glu-Xaa- | |
| | Val-Xaa-Glu-Xaa-Ala-Xaa-Xaa-Xaa-Gly-Xaa- | |
| | Xaa-Val-Xaa-Xaa-Pro-Asp-Asn-Phe-Tyr- | |
| | Xaa-Leu-Val-Ala-Xaa-Xaa-Xaa-Glu-Xaa-Xaa | |
| | (SEQ ID NO: 34) | |
| 50% | Formula III: | 90% |
| | Met-Xaa-Met-Asp-Met-Xaa-Thr-Arg-Met- | |
| | Phe-Lys-Glu-Glu-Gly-Trp-Ile-Arg-Lys-Xaa- | |
| | Cys-Lys-Xaa-Cys-Gly-Lys-Xaa-Phe-Trp- | |
| | Thr-Leu-Asp-Pro-Asp-Arg-Glu-Thr-Cys-Gly- | |
| | Asp-Pro-Pro-Cys-Asp-Glu-Tyr-Xaa-Phe-Ile- | |
| | Gly-Lys-Pro-Gly-Ile-Pro-Lys-Lys-Tyr-Thr- | |
| | Leu-Xaa-Glu-Met-Arg-Glu-Lys-Phe-Leu-Ser- | |
| | Phe-Phe-Glu-Xaa-Xaa-Gly-His-Gly-Arg-Val- | |
| | Lys-Arg-Tyr-Pro-Val-Leu-Pro-Arg-Trp-Arg- | |
| | Asp-Asp-Val-Leu-Leu-Val-Gly-Ala-Ser-Ile- | |
| | Met-Asp-Phe-Gln-Pro-Trp-Val-Ile-Ser-Gly- | |
| | Glu-Ala-Asp-Pro-Pro-Ala-Asn-Pro-Leu-Thr-Ile- | |
| | Ser-Gln-Pro-Ser-Ile-Arg-Phe-Thr-Asp-Ile-Asp- | |
| | Asn-Val-Gly-Ile-Thr-Gly-Arg-His-Phe- | |
| | Thr-Ile-Phe-Glu-Met-Met-Ala-His-His- | |
| | Ala-Phe-Asn-Tyr-Pro-Gly-Lys-Pro-Ile- | |
| | Tyr-Trp-Met-Asp-Glu-Thr-Val-Glu- | |
| | Leu-Ala-Phe-Glu-Phe-Phe-Thr-Lys-Xaa- | |
| | Leu-Gly-Met-Lys-Pro-Glu-Asp- | |
| | Ile-Thr-Phe-Lys-Glu-Asn-Pro- | |
| | Trp-Ala-Gly-Gly-Gly-Asn-Ala-Gly- | |
| | Pro-Ala-Phe-Glu-Val-Leu-Tyr-Arg-Gly-Leu- | |
| | Glu-Val-Ala-Thr-Leu-Val-Phe-Met-Gln- | |
| | Tyr-Lys-Xaa-Ala-Pro-Xaa-Xaa-Ala-Xaa- | |
| | Xaa-Xaa-Gln-Val-Val-Xaa-Ile-Lys-Gly- | |
| | Asp-Xaa-Tyr-Val-Pro-Met-Xaa-Thr-Xaa- | |
| | Val-Val-Asp-Thr-Gly-Tyr-Gly- | |
| | Leu-Glu-Arg-Leu-Val-Trp-Met-Ser-Gln-Gly- | |
| | Thr-Pro-Thr-Ala-Tyr-Asp-Ala-Val- | |
| | Leu-Gly-Tyr-Val-Val-Glu-Pro-Leu-Lys- | |
| | Xaa-Met-Ala-Gly-Xaa- | |
| | Glu-Lys-Ile-Asp-Xaa-Xaa-Ile-Leu-Met- | |
| | Glu-Asn-Ser-Arg-Leu-Ala-Gly-Met-Phe-Asp- | |
| | Ile-Glu-Asp-Met-Gly-Asp-Leu-Arg-Xaa- | |
| | Leu-Arg-Xaa-Xaa-Val-Ala- | |
| | Xaa-Arg-Val-Gly-Ile-Ser-Val-Glu-Glu-Leu- | |
| | Glu-Xaa-Xaa-Xaa-Arg-Pro-Tyr-Glu-Leu-Ile- | |
| | Tyr-Ala-Ile-Ala-Asp-His-Thr-Lys-Ala- | |
| | Leu-Thr-Phe-Met-Leu-Ala-Asp-Gly- | |
| | Val-Ile-Pro-Ser-Asn-Val-Lys-Ala-Gly- | |
| | Tyr-Leu-Ala-Arg-Leu-Leu-Ile-Arg-Lys- | |
| | Ser-Ile-Arg-His-Leu-Arg-Glu-Leu- | |
| | Gly-Leu-Glu-Xaa-Pro-Leu-Ser- | |
| | Glu-Ile-Val-Ala-Met-His-Ile-Lys-Glu-Leu- | |
| | Xaa-Xaa-Thr-Phe-Pro-Glu-Phe-Lys- | |
| | Glu-Met-Glu-Asp-Val-Ile-Leu-Asp-Ile-Xaa- | |
| | Xaa-Val-Glu-Glu-Lys-Arg-Tyr-Xaa-Glu-Thr- | |
| | Leu-Xaa-Arg-Gly-Ser-Xaa-Leu-Val- | |
| | Xaa-Arg-Glu-Ile-Xaa-Lys-Leu-Lys- | |
| | Lys-Xaa-Gly-Xaa-Xaa-Glu-Xaa-Pro-Leu-Glu- | |
| | Lys-Leu-Ile-Leu-Phe-Tyr-Glu-Ser-His-Gly- | |
| | Leu-Thr-Pro-Glu-Ile-Val-Xaa-Glu-Ile-Ala-Glu- | |
| | Lys-Glu-Gly-Xaa-Lys-Val-Xaa-Ile-Pro- | |
| | Asp-Asn-Phe-Tyr-Ser-Leu-Val-Ala-Lys-Xaa- | |
| | Ala-Glu-Xaa-Xaa (SEQ ID NO: 35) | |

¹Xaa designates any amino acid

A claimed AMRS may be defined in terms of a consensus sequence, e.g. as a variant of a consensus sequence. A claimed AMRS, which is defined as a variant of a consensus sequence, may be further defined by a specified level of sequence identity between the conserved amino acids (i.e. amino acids not designated Xaa) of the consensus sequence and a reference sequence. In this case it is to be understood that the claimed variant of the consensus sequence may have variation in the conserved amino acid positions to the extent that it fails within the limits set by the specified sequence identity between the consensus sequence and the reference sequence. A non-limiting example of a claimed AMRS, defined as a variant of a consensus sequence, is provided in the following:
A. A 2-Aminoisobutyric acid-tRNA synthetase (AibRS) comprising an amino acid sequence of SEQ ID NO: 7 or a variant hereof, wherein the variant of SEQ ID NO: 7 comprises 215Gly.
B. The AibRS according to claim A; wherein the variant of SEQ ID NO: 7 is a variant of Formula I; wherein Formula I is:

(SEQ ID NO: 33)
Xaa-Xaa-Xaa-Xaa-Xaa-Xaa-Xaa-Xaa-Xaa-Phe-Xaa-Glu-
Xaa-Gly-Xaa-Xaa-Xaa-Lys-Xaa-Cys-Xaa-Xaa-Cys-Gly-
Xaa-Xaa-Phe-Trp-Thr-Xaa-Xaa-Xaa-Xaa-Arg-Xaa-Xaa-
Cys-Gly-Asp-Xaa-Pro-Cys-Xaa-Xaa-Tyr-Xaa-Phe-Ile-
Gly-Xaa-Pro-Xaa-Xaa-Xaa-Xaa-Xaa-Xaa-Xaa-Leu-Xaa-
Glu-Xaa-Arg-Xaa-Xaa-Phe-Xaa-Xaa-Phe-Phe-Glu-Xaa-
Xaa-Xaa-His-Xaa-Xaa-Xaa-Xaa-Arg-Tyr-Pro-Val-Xaa-
Xaa-Arg-Trp-Arg-Asp-Asp-Val-Xaa-Leu-Val-Gly-Ala-
Ser-Ile-Xaa-Asp-Phe-Gln-Pro-Trp-Val-Xaa-Xaa-Gly-
Xaa-Xaa-Xaa-Pro-Pro-Ala-Asn-Pro-Leu-Xaa-Ile-Ser-
Gln-Pro-Xaa-Ile-Arg-Xaa-Xaa-Asp-Xaa-Asp-Xaa-Val-
Gly-Xaa-Xaa-Gly-Arg-His-Xaa-Thr-Xaa-Phe-Glu-Met-
Met-Ala-His-His-Ala-Phe-Asn-Xaa-Xaa-Xaa-Xaa-Xaa-
Xaa-Tyr-Trp-Xaa-Xaa-Glu-Thr-Val-Xaa-Xaa-Xaa-Xaa-
Xaa-Phe-Xaa-Xaa-Xaa-Xaa-Xaa-Xaa-Xaa-Xaa-Xaa-Xaa-
Xaa-Ile-Thr-Phe-Xaa-Glu-Xaa-Xaa-Trp-Xaa-Gly-Gly-
Gly-Asn-Ala-Gly-Xaa-Xaa-Xaa-Glu-Val-Xaa-Xaa-Xaa-
Gly-Xaa-Glu-Xaa-Ala-Thr-Leu-Val-Phe-Met-Xaa-Tyr-
Xaa-Xaa-Xaa-Xaa-Xaa-Xaa-Xaa-Xaa-Xaa-Xaa-Xaa-Xaa-
Xaa-Xaa-Xaa-Xaa-Xaa-Xaa-Xaa-Xaa-Xaa-Pro-Xaa-Xaa-
Xaa-Xaa-Xaa-Val-Asp-Thr-Gly-Tyr-Gly-Leu-Glu-Arg-
Xaa-Xaa-Trp-Xaa-Ser-Xaa-Gly-Xaa-Pro-Thr-Xaa-Tyr-
Asp-Ala-Xaa-Xaa-Xaa-Xaa-Xaa-Xaa-Xaa-Xaa-Leu-Xaa-
Xaa-Xaa-Ala-Gly-Xaa-Xaa-Xaa-Xaa-Xaa-Xaa-Xaa-Xaa-
Ile-Leu-Xaa-Xaa-Xaa-Xaa-Xaa-Xaa-Ala-Gly-Xaa-Xaa-
Asp-Xaa-Xaa-Xaa-Xaa-Xaa-Xaa-Leu-Xaa-Xaa-Leu-Arg-
Xaa-Xaa-Val-Ala-Xaa-Xaa-Xaa-Xaa-Xaa-Xaa-Xaa-Xaa-
Glu-Leu-Xaa-Xaa-Xaa-Xaa-Xaa-Pro-Xaa-Glu-Xaa-Xaa-
Tyr-Ala-Ile-Ala-Asp-His-Thr-Xaa-Xaa-Leu-Xaa-Phe-
Met-Leu-Xaa-Asp-Gly-Val-Xaa-Pro-Ser-Asn-Xaa-Xaa-
Ala-Gly-Tyr-Leu-Ala-Arg-Leu-Xaa-Ile-Arg-Xaa-Xaa-
Xaa-Arg-Xaa-Xaa-Xaa-Xaa-Leu-Gly-Xaa-Xaa-Xaa-Pro-
Leu-Xaa-Xaa-Ile-Xaa-Xaa-Xaa-Xaa-Xaa-Xaa-Xaa-Xaa-
Xaa-Xaa-Xaa-Xaa-Pro-Glu-Xaa-Xaa-Glu-Xaa-Xaa-Xaa-
Xaa-Xaa-Xaa-Xaa-Xaa-Xaa-Xaa-Xaa-Glu-Glu-Xaa-Xaa-
Xaa-Xaa-Xaa-Thr-Xaa-Xaa-Arg-Gly-Xaa-Xaa-Xaa-Xaa-
Xaa-Xaa-Xaa-Xaa-Xaa-Xaa-Xaa-Lys-Xaa-Xaa-Gly-Xaa-
Xaa-Xaa-Xaa-Pro-Xaa-Xaa-Xaa-Leu-Xaa-Xaa-Xaa-Tyr-
Xaa-Ser-His-Gly-Xaa-Xaa-Pro-Glu-Xaa-Xaa-Xaa-Glu-
Xaa-Ala-Xaa-Xaa-Xaa-Xaa-Xaa-Xaa-Val-Xaa-Xaa-Pro-
Asp-Asn-Phe-Tyr-Xaa-Xaa-Val-Ala-Xaa-Xaa-Xaa-Xaa-
Xaa-Xaa

wherein each Xaa is independently selected and is one or more amino acids, or absent; wherein the variant of Formula I is at least 90% identical to SEQ ID NO: 7 in positions not designated Xaa.

In this example the claim language of claim B defines the claimed AibRS as a variant of a consensus sequence, i.e. a variant of Formula I. Further, the claim language of claim B defines that the variant of Formula I is at least 90% identical to SEQ ID NO: 7 in positions not designated Xaa, meaning that 10% variation, when compared to SEQ ID NO: 7, is allowed in the conserved amino acids. According to Claim A, on which Claim B depends, position 215 must be Gly. Formula I defines position 215 to be Val, however, since the claimed AibRS of claim B is defined as a variant of Formula I (and not as Formula I as such) variation in the conserved amino acids are allowed as explained above, and hence the claimed variant of Formula I may contain Gly in position 215, and consequently there is no contradiction between Claims A and B.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-14. (canceled)

15. A method of preparing a polypeptide containing one or more Aib residues using a 2-aminoisobutyric acid-tRNA synthetase (AibRS), wherein the AibRS comprises an amino acid sequence of SEQ ID NO: 7 or a variant thereof and having the function of aminoacylating tRNA with 2-aminoisobutyric acid (Aib), wherein the variant of SEQ ID NO: 7 retains 215Gly and is at least 90% identical to SEQ ID NO: 7.

16. The method according to claim 15, wherein the AibRS variant of SEQ ID NO: 7 comprises the following:
position 192 is selected from Trp, His, Val, Ile, or Leu,
position 193 is selected from Ala, Leu, Ile, or Gly,
position 213 is selected from Thr, Ser, Cys, or Ala,
position 216 is selected from Phe or Trp,
position 217 is selected from Met, Ile, or Leu,
position 249 is selected from Thr, Ser, Val, or Phe,
position 360 is selected from Asn or Ala, and
position 459 is selected from Glu or Ala.

17. The method according to claim 15, wherein the AibRS variant of SEQ ID NO: 7 comprises: [192His; 215Gly]; [192His; 215Gly; 360Ala; 459Ala], [192His; 215Gly; 217Ile; 360Ala; 459Ala], [192His; 215Gly; 217Leu; 360Ala; 459Ala], [192His; 215Gly; 193Leu; 360Ala; 459Ala], [192His; 215Gly; 216Trp; 360Ala; 459Ala], [192His; 215Gly; 193Leu; 216Trp; 360Ala; 459Ala], [192His; 215Gly; 193Ile; 360Ala; 459Ala], [192His; 215Gly; 193Ile; 217Ile; 360Ala; 459Ala ], [192His; 215Gly; 193Leu; 217Ile; 360Ala; 459Ala ], [192His; 215Gly; 193Ile; 217Leu; 360Ala, 459Ala ], [192His; 215Gly; 193Leu; 217Leu; 360Ala; 459Ala ], [192Val; 215Gly; 217Ile; 360Ala; 459Ala ], [192Ile; 215Gly; 217Ile; 360Ala; 459Ala], [192Leu; 215Gly; 217Ile; 360Ala; 459Ala], [192His; 193Gly; 215Gly; 217Ile; 360Ala; 459Ala ], [192His; 213Ser; 215Gly; 360Ala; 459Ala ], [192His; 215Gly; 249Ser; 360Ala; 459Ala], [192His; 215Gly; 249Val; 360Ala; 459Ala], [192His; 213Cys; 215Gly; 249Val; 360Ala; 459Ala], or [192His; 213Ala; 215Gly; 249Phe; 360Ala; 459Ala].

18. The method according to claim 17, wherein the AibRS variant of SEQ ID NO: 7 is selected from a list consisting of: SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, SEQ ID NO: 26, SEQ ID NO: 27, SEQ ID NO: 28, SEQ ID NO: 29, and SEQ ID NO: 30.

19. The method according to claim 17, wherein the tRNA is a suppressor tRNA.
